(19) United States
(12) Patent Application Publication
(10) Pub. No.: US 2023/0355978 A1
(43) Pub. Date: Nov. 9, 2023

(54) Duty Cycling Control of Neural Dose in a Spinal Cord Stimulator System

(71) Applicant: Boston Scientific Neuromodulation Corporation, Valencia, CA (US)

(72) Inventors: Ismael Huertas Fernandez, Sevilla (ES); Que T. Doan, West Hills, CA (US)

(21) Appl. No.: 18/298,024

(22) Filed: Apr. 10, 2023

Related U.S. Application Data
(60) Provisional application No. 63/364,394, filed on May 9, 2022.

(51) Int. Cl. A61N 1/36; A61N 1/372

(52) U.S. Cl. CPC A61N 1/36062; A61N 1/36175; A61N 1/37247

(57) ABSTRACT

Systems and methods for controlling an implantable stimulator device such as a spinal cord stimulation are disclosed. Optimal stimulation parameters for the patient, which may be personalized for the patient based on test stimulation and organized as a neural dose, are subject to duty cycling in which stimulation is applied during an on duration and is turned off during an off duration. The duty cycling applied is automatically determined by and dependent on the stimulation parameters chosen, and in particular the frequency of those parameters. Further, a user interface element is provided to allow a user to automatically step thought suitable duty cycle values determined to be appropriate for the stimulation parameters. As such, the user does need not need to engage in guess work to specify individual on and off times when duty cycling is desired.

### CROSS REFERENCE TO RELATED APPLICATIONS

This is a non-provisional application of U.S. Provisional Patent Application Ser. No. 63/364,394, filed May 9, 2022, which is incorporated herein by reference in its entirety, and to which priority is claimed.

### FIELD OF THE INVENTION

This application relates to Implantable Medical Devices (IMDs) generally, Spinal Cord Stimulators more specifically, and to methods of control of such devices.

### INTRODUCTION

Implantable neurostimulator devices are devices that generate and deliver electrical stimuli to body nerves and tissues for the therapy of various biological disorders, such as pacemakers to treat cardiac arrhythmia, defibrillators to treat cardiac fibrillation, cochlear stimulators to treat deafness, retinal stimulators to treat blindness, muscle stimulators to produce coordinated limb movement, spinal cord stimulators to treat chronic pain, cortical and deep brain stimulators to treat motor and psychological disorders, and other neural stimulators to treat urinary incontinence, sleep apnea, shoulder subluxation, etc. The description that follows will generally focus on the use of the invention within a Spinal Cord Stimulation (SCS) system, such as that disclosed in U.S. Pat. No. 6,516,227. However, the present invention may find applicability with any implantable neurostimulator device system.

An SCS system typically includes an Implantable Pulse Generator (IPG) 10 shown in FIG. 1. The IPG 10 includes a biocompatible device case 12 that holds the circuitry and battery 14 necessary for the IPG to function. The IPG 10 is coupled to electrodes 16 via one or more electrode leads 15 that form an electrode array 17. The electrodes 16 are configured to contact a patient's tissue and are carried on a flexible body 18, which also houses the individual lead wires 20 coupled to each electrode 16. The lead wires 20 are also coupled to proximal contacts 22, which are insertable into lead connectors 24 fixed in a header 23 on the IPG 10, which header can comprise an epoxy for example. Once inserted, the proximal contacts 22 connect to header contacts within the lead connectors 24, which are in turn coupled by feedthrough pins through a case feedthrough to circuitry within the case 12, although these details aren't shown.

In the illustrated IPG 10, there are sixteen lead electrodes (E1-E16) split between two leads 15, with the header 23 containing a 2×1 array of lead connectors 24. However, the number of leads and electrodes in an IPG is application specific and therefore can vary. The conductive case 12 can also comprise an electrode (Ec). In a SCS application, the electrode leads 15 are typically implanted proximate to the dura in a patient's spinal column on the right and left sides of the spinal cord midline. The proximal electrodes 22 are tunneled through the patient's tissue to a distant location such as the buttocks where the IPG case 12 is implanted, at which point they are coupled to the lead connectors 24. In other IPG examples designed for implantation directly at a site requiring stimulation, the IPG can be lead-less, having electrodes 16 instead appearing on the body of the IPG for contacting the patient's tissue. The IPG leads 15 can be integrated with and permanently connected the case 12 in other IPG solutions. The goal of SCS therapy is to provide electrical stimulation from the electrodes 16 to alleviate a patient's symptoms, most notably chronic back pain. IPG 10 as disclosed herein may also comprise an External Trial Stimulator (ETS), which generally mimics operation of the IPG, but resides externally in communication with implanted leads. See, e.g., 9,259,574, disclosing a design for an ETS.

IPG 10 can include an antenna 26a allowing it to communicate bi-directionally with a number of external systems, shown later in FIG. 4. The antenna 26a as depicted in FIG. 1 is shown as a conductive coil within the case 12, although the coil antenna 26a can also appear in the header 23. When antenna 26a is configured as a coil, communication with external devices preferably occurs using near-field magnetic induction. IPG may also include a Radio-Frequency (RF) antenna 26b. In FIG. 1, RF antenna 26b is shown within the header 23, but it may also be within the case 12. RF antenna 26b may comprise a patch, slot, or wire, and may operate as a monopole or dipole. RF antenna 26b preferably communicates using far-field electromagnetic waves. RF antenna 26b may operate in accordance with any number of known RF communication standards, such as Bluetooth, Zigbee, WiFi, MICS, and the like.

Stimulation in IPG 10 is typically provided by pulses, as shown in FIG. 2. Stimulation parameters typically include the amplitude of the pulses (A; whether current or voltage); the frequency (F) and pulse width (PW) of the pulses; the electrodes 16 (E) activated to provide such stimulation; and the polarity (P) of such active electrodes, i.e., whether active electrodes are to act as anodes (that source current to the tissue) or cathodes (that sink current from the tissue). These are possibly other stimulation parameters taken together comprise a stimulation program that the IPG 10 can execute to provide therapeutic stimulation to a patient.

In the example of FIG. 2, electrode E5 has been selected as an anode, and thus provides pulses which source a positive current of amplitude +A to the tissue. Electrode E4 has been selected as a cathode, and thus provides pulses which sink a corresponding negative current of amplitude-A from the tissue. This is an example of bipolar stimulation, in which only lead-based electrodes are used to provide stimulation to the tissue (one anode, one cathode). However, more than one electrode may act as an anode at a given time, and more than one electrode may act as a cathode at a given time. The case electrode Ec may also be selected, providing monopolar stimulation.

The pulses as shown in FIG. 2 are biphasic, comprising a first phase 30a, followed quickly thereafter by a second phase 30b of opposite polarity. As is known, use of a biphasic pulse is useful in active charge recovery. For example, each electrodes' current path to the tissue may include a serially-connected DC-blocking capacitor (38, FIG. 3), which will charge during the first phase 30a and discharge (be recovered) during the second phase 30b. In the example shown, the first and second phases 30a and 30b have the same duration and amplitude (although opposite polarities), which ensures the same amount of charge during both phases. However, the second phase 30b may also be charged balance with the first phase 30a if the integral of the amplitude and durations of the two phases are equal in magnitude, as is well known. The width of each pulse, PW, is defined here as the duration of first pulse phase 30a, although pulse width could also refer to the total duration of the first and second pulse phases 30a and 30b as well. Note that an interphase period (IP) during which no stimulation is provided may be provided between the two phases 30a and 30b.

IPG 10 includes stimulation circuitry 28 that can be programmed to produce the stimulation pulses at the electrodes as defined by the stimulation program, and an example of such circuitry is shown in FIG. 3. In this example, the stimulation circuitry 28 can control the current or charge at each electrode independently. The stimulation circuitry 28 includes one or more current sources (PDACi) for sourcing current to the tissue and one or more current sinks (NDACi) for sinking current from the tissue. These sources and sinks can comprise Digital-to-Analog converters (DACs). In the example shown, a PDAC/NDAC pair is dedicated (hardwired) to a particular electrode node ei 39. Each electrode node ei 39 is preferably connected to an electrode Ei 16 via a DC-blocking capacitor Ci 38, which acts as a safety measure to prevent DC current injection into the patient, as could occur for example if there is a circuit fault in the stimulation circuitry 28. The PDACs and NDACs are preferably current sources, but could comprise voltage sources as well.

Proper control of the PDACs and NDACs allows any of the electrodes 16 and the case electrode Ec 12 to act as anodes or cathodes to create a current through a patient's tissue. Such control preferably comes in the form of digital signals Iip and Iin that set the anodic and cathodic current at each electrode Ei. If for example it is desired to set electrode E1 as an anode with a current of +3 mA, and to set electrodes E2 and E3 as cathodes with a current of −1.5 mA each, control signal I1p would be set to the digital equivalent of 3 mA to cause PDAC1 to produce +3 mA, and control signals I2n and I3n would be set to the digital equivalent of 1.5 mA to cause NDAC2 and NDAC3 to each produce −1.5 mA. Note that definition of these control signals can also occur using a single programmed amplitude A and percentage X % for each, as explained further later with reference to FIG. 5. For example, amplitude A may be set to 3 mA, with E1 designated as an anode with X=100%, and with E2 and E3 designated as cathodes with X=50%.

Stimulation circuitry 28 can differ in design, and different examples are described in U.S. Pat. Nos. 6,181,969, 8,606,362, 8,620,436, and U.S. Patent Application Publications 2018/0071513, 2018/0071520, and 2019/0083796. Some stimulation circuitries 28 can include switching matrices that can intervene between the one or more PDACs and the electrode nodes ei 39, and between the one or more NDACs and the electrode nodes, which allows one or more of the PDACs or one or more of the NDACs to be connected to one or more electrode nodes at a given time. Stimulation circuitry 28 can be integrated on one or more Application Specific Integrated Circuits (ASICs), as described in U.S. Patent Application Publications 2012/0095529, 2012/0092031, and 2012/0095519. As explained in these references, ASIC(s) may also contain other circuitry useful in the IPG 10, such as telemetry circuitry (for interfacing off chip with the IPG's telemetry antennas), circuitry for generating the compliance voltage VH that powers the stimulation circuitry, various measurement circuits, etc.

FIG. 4 shows various external systems 60, 70, and 80 that can wirelessly communicate data with the IPG 10. Such systems can be used to wirelessly transmit a stimulation program to the IPG 10—that is, to program its stimulation circuitry 28 to produce stimulation with desired amplitudes and timings as described earlier. Such systems may also be used to adjust one or more stimulation parameters of a stimulation program that the IPG 10 is currently executing, and/or to wirelessly receive information from the IPG 10, such as various status information, etc.

External controller 60 can be as described in U.S. Patent Application Publication 2015/0080982 for example, and may comprise a portable, hand-held controller dedicated to work with the IPG 10. External controller 60 may also comprise a general-purpose mobile electronics device such as a mobile phone which has been programmed with a Medical Device Application (MDA) allowing it to work as a wireless controller for the IPG 10, as described in U.S. Patent Application Publication 2015/0231402. External controller 60 includes a display 61 and a means for entering commands, such as buttons 62 or selectable graphical icons provided on the display 61. The external controller 60's user interface enables a patient to adjust stimulation parameters, although it may have limited functionality when compared to systems 70 and 80, described shortly. The external controller 60 can have one or more antennas capable of communicating with the IPG 10. For example, the external controller 60 can have a near-field magnetic-induction coil antenna 64a capable of wirelessly communicating with the coil antenna 26a in the IPG 10. The external controller 60 can also have a far-field RF antenna 64b capable of wirelessly communicating with the RF antenna 26b in the IPG 10.

Clinician programmer 70 is described further in U.S. Patent Application Publication 2015/0360038, and can comprise a computing device such as a desktop, laptop, or notebook computer, a tablet, a mobile smart phone, a Personal Data Assistant (PDA)-type mobile computing device, etc. In FIG. 4, the computing device is shown as a laptop computer that includes typical computer user interface means such as a display 71, buttons 72, as well as other user-interface devices such as a mouse, a keyboard, speakers, a stylus, a printer, etc., not all of which are shown for convenience. Also shown in FIG. 4 are accessory devices for the clinician programmer 70 that are usually specific to its operation as a stimulation controller, such as a communication “wand” 76 coupleable to suitable ports on the computing device. The antenna used in the clinician programmer 70 to communicate with the IPG 10 can depend on the type of antennas included in the IPG 10. If the patient's IPG 10 includes a coil antenna 26a, wand 76 can likewise include a coil antenna 74a to establish near-field magnetic-induction communications at small distances. In this instance, the wand 76 may be affixed in close proximity to the patient, such as by placing the wand 76 in a belt or holster wearable by the patient and proximate to the patient's IPG 10. If the IPG 10 includes an RF antenna 26b, the wand 76, the computing device, or both, can likewise include an RF antenna 74b to establish communication with the IPG 10 at larger distances. The clinician programmer 70 can also communicate with other devices and networks, such as the Internet, either wirelessly or via a wired link provided at an Ethernet or network port.

External system 80 comprises another means of communicating with and controlling the IPG 10 via a network 85 which can include the Internet. The network 85 can include a server 86 programmed with communication and control functionality, and may include other communication networks or links such as WiFi, cellular or land-line phone links, etc. The network 85 ultimately connects to an intermediary device 82 having antennas suitable for communication with the IPG's antenna, such as a near-field magnetic-induction coil antenna 84a and/or a far-field RF antenna 84b. Intermediary device 82 may be located generally proximate to the IPG 10. Network 85 can be accessed by any user terminal 87, which typically comprises a computer device associated with a display 88. External system 80 allows a remote user at terminal 87 to communicate with and control the IPG 10 via the intermediary device 82.

FIG. 4 also shows circuitry 90 involved in any of external systems 60, 70, or 80. Such circuitry can include control circuitry 92, which can comprise any number of devices such as one or more microprocessors, microcomputers, FPGAs, DSPs, other digital logic structures, etc., which are capable of executing programs in a computing device. Such control circuitry 92 may contain or coupled with memory 94 which can store external system software 96 for controlling and communicating with the IPG 10, and for rendering a Graphical User Interface (GUI) 100 on a display (61, 71, 88) associated with the external system. In external system 80, the external system software 96 would likely reside in the server 86, while the control circuitry 92 could be present in either or both the server 86 or the terminal 87.

A portion of a typical GUI 100 for controlling operation of the IPG 10 is shown in one example in FIG. 5. One skilled in the art will understand that the particulars of the GUI 100 will depend on where software 96 is in its execution, which will depend on the GUI selections the clinician has made. FIG. 5 shows the GUI 100 at a point allowing for the setting of stimulation parameters for the patient and for their storage as a stimulation program. To the left a program interface 102 is shown, which as explained further in the '038 Publication allows for naming, loading and saving of stimulation programs for the patient. Shown to the right is a stimulation parameters interface 104, in which specific stimulation parameters (A, PW, F, E, P, X) can be defined for a stimulation program. Values for stimulation parameters relating to the shape of the waveform (A; in this example, current), pulse width (PW), and frequency (F) are shown in a waveform parameter interface 106, including buttons the clinician can use to increase or decrease these values. A cursor 110 (or other selection means such as a mouse pointer) can be used to select various options presented by the GUI 100.

Stimulation parameters relating to the electrodes 16 (the electrodes E activated and their polarities P), are made adjustable in an electrode parameter interface 108. A leads interface 104 displays the leads 15 of the electrode array 17 in their proper position with respect to each other, for example, on the left and right sides of the spinal column. Buttons in the electrode parameter interface 108 allow an electrode (including the case electrode, Ec) to be designated as an anode, a cathode, or off. The electrode parameter interface 108 further allows the relative strength of anodic or cathodic current (A) of a selected electrode to be specified in terms of a percentage, X %. This is particularly useful if more than one electrode is to act as an anode or cathode at a given time, as explained above.

Once stimulation parameters have been selected, the leads interface 104 can show a depiction of the resulting poles 112, which can include one or more anode poles (+) and one or more cathode poles (−). These poles 112 may be displayed as they are formed during a particular pulse phase (such as 30a, FIG. 2). Notice that by splitting the anodic and cathodic current between different electrodes, the physical position of the poles 112 may not necessarily correspond to the physical positions of the electrodes, as explained further in U.S. Pat. No. 10,881,859, which is incorporated herein by reference. As also explained in the '859 patent, the poles 112 can also be selected and moved in the electrode array, with the software 96 making necessary adjustments to the stimulation parameters to position the poles at the desired positions.

### SUMMARY

A method is disclosed for controlling an implantable stimulator device of a patient using an external device in communication with the implantable stimulator device. The method may comprise: receiving at a graphical user interface (GUI) of the external device a selection of a set of stimulation parameters for the implantable stimulator device to execute to provide stimulation to the patient; and providing on the GUI a first interface to select from one of a plurality of duty cycles for the stimulation, wherein each duty cycle comprises an on duration and an off duration at which the stimulation will be repeatedly enabled and disabled, wherein the plurality of selectable duty cycles are dependent on the selected set of stimulation parameters.

In one example, the selected set of stimulation parameters defines the stimulation as a sequence of stimulation pulses. In one example, the selected set of stimulation parameters comprises a frequency of the stimulation pulses. In one example, the plurality of selectable duty cycles are dependent on the frequency of the selected set of stimulation parameters. In one example, the plurality of selectable duty cycles have values that decrease as the frequency of the selected set of stimulation parameters increases. In one example, the first interface is configured to allow a user to step though the plurality of duty cycles. In one example, the first interface is configured to step through the plurality of duty cycles from a minimum duty cycle to a maximum duty cycle. In one example, the minimum duty cycle is defined by a minimum charge rate, and wherein the maximum duty cycle is defined by a maximum charge rate. In one example, the minimum charge rate and the maximum charge rate are expressed in microCoulombs per second, and respectively comprise 2.5 and 20, or 8 and 80, or 12 and 140, or 15 or 200, or 18.9 and 294, or 18.8 and 346, or 20 and 450. In one example, the minimum charge rate and the maximum charge comprise 2.5 and 20 if a frequency of the set of stimulation parameters comprises 10 Hz, or 8 and 80 if a frequency of the set of stimulation parameters comprises 50 Hz, or 12 and 140 if a frequency of the set of stimulation parameters comprises 100 Hz, or 15 or 200 if a frequency of the set of stimulation parameters comprises 200 Hz, or 18.9 and 294 if a frequency of the set of stimulation parameters comprises 600 Hz, or 18.8 and 346 if a frequency of the set of stimulation parameters comprises 10 Hz, or 20 and 450 if a frequency of if a frequency of set of stimulation parameters comprises 10 Hz set of stimulation parameters comprises 1000 Hz. In one example, the plurality of duty cycles are defined using duration parameters including a minimum on duration, a maximum on duration, a minimum off duration, and a maximum off duration, wherein the duration parameters are dependent on the selected set of stimulation parameters. In one example, the plurality of duty cycles are defined using different combinations of on durations bounded between the minimum and maximum on durations, and off durations bounded between the minimum and maximum off durations. In one example, the selection of the set of stimulation parameters is received at a second interface provided on the GUI. In one example, the second interface is configured to allow a user to step though a plurality of sets of stimulation parameters to select the set of stimulation parameters. In one example, each of the plurality of sets of stimulation parameters comprises a frequency, an amplitude, and a pulse width of the stimulation. In one example, the plurality of sets of stimulation parameters are derived from a model for the patient determined from providing test stimulation to the patient. In one example, the method further comprises providing the test stimulation to the patient to determine the model. In one example, each of the sets of stimulation parameters comprises a charge rate, wherein the second interface is configured to step through the plurality of sets of stimulation parameters from a minimum charge rate to a maximum charge rate. In one example, each of the sets of stimulation parameters comprises a charge rate, wherein the second interface is configured to step through the plurality of sets of stimulation parameters from a minimum charge rate to a maximum charge rate. In one example, the method further comprises receiving at the GUI at least one indication of a response from the patient to the stimulation. In one example, the plurality of selectable duty cycles are further dependent on the at least one indication of the response. In one example, the at least one indication of the response comprises one or more of a perception threshold, a pain score, a pain coverage metric, or a measured neural response to the stimulation. In one example, the stimulation comprises sub-perception stimulation.

An external device is disclosed for controlling an implantable stimulator device of a patient, which may comprise: a graphical user interface (GUI) configured to allow a user to select a set of stimulation parameters for the implantable stimulator device to execute to provide stimulation to the patient; wherein the GUI comprises a first interface configured to allow the user to select from one of a plurality of duty cycles for the stimulation, wherein each duty cycle comprises an on duration and an off duration at which the stimulation will be repeatedly enabled and disabled, wherein the plurality of selectable duty cycles are dependent on the selected set of stimulation parameters.

In one example, the selected set of stimulation parameters defines the stimulation as a sequence of stimulation pulses. In one example, the selected set of stimulation parameters comprises a frequency of the stimulation pulses. In one example, the plurality of selectable duty cycles are dependent on the frequency of the selected set of stimulation parameters. In one example, the plurality of selectable duty cycles have values that decrease as the frequency of the selected set of stimulation parameters increases. In one example, the first interface is configured to allow a user to step though the plurality of duty cycles. In one example, the first interface is configured to step through the plurality of duty cycles from a minimum duty cycle to a maximum duty cycle. In one example, the minimum duty cycle is defined by a minimum charge rate, and wherein the maximum duty cycle is defined by a maximum charge rate. In one example, the minimum charge rate and the maximum charge rate are expressed in microCoulombs per second, and respectively comprise 2.5 and 20, or 8 and 80, or 12 and 140, or 15 or 200, or 18.9 and 294, or 18.8 and 346, or 20 and 450. In one example, the minimum charge rate and the maximum charge comprise 2.5 and 20 if a frequency of the set of stimulation parameters comprises 10 Hz, or 8 and 80 if a frequency of the set of stimulation parameters comprises 50 Hz, or 12 and 140 if a frequency of the set of stimulation parameters comprises 100 Hz, or 15 or 200 if a frequency of the set of stimulation parameters comprises 200 Hz, or 18.9 and 294 if a frequency of the set of stimulation parameters comprises 600 Hz, or 18.8 and 346 if a frequency of the set of stimulation parameters comprises 10 Hz, or 20 and 450 if a frequency of if a frequency of set of stimulation parameters comprises 10 Hz set of stimulation parameters comprises 1000 Hz. In one example, the plurality of duty cycles are defined using duration parameters including a minimum on duration, a maximum on duration, a minimum off duration, and a maximum off duration, wherein the duration parameters are dependent on the selected set of stimulation parameters. In one example, the plurality of duty cycles are defined using different combinations of on durations bounded between the minimum and maximum on durations, and off durations bounded between the minimum and maximum off durations. In one example, the GUI further comprises a second interface configured to allow the user to select the set of stimulation parameters. In one example, the second interface is configured to allow a user to step though a plurality of sets of stimulation parameters to select the set of stimulation parameters. In one example, each of the plurality of sets of stimulation parameters comprises a frequency, an amplitude, and a pulse width of the stimulation. In one example, the plurality of sets of stimulation parameters are derived from a model stored in the external device. In one example, each of the sets of stimulation parameters comprises a charge rate, wherein the second interface is configured to step through the plurality of sets of stimulation parameters from a minimum charge rate to a maximum charge rate. In one example, each of the sets of stimulation parameters comprises a charge rate, wherein the second interface is configured to step through the plurality of sets of stimulation parameters from a minimum charge rate to a maximum charge rate. In one example, the plurality of sets of stimulation parameters are configured to provide sub-perception stimulation for the patient. In one example, the GUI is further configured to receive at least one indication of a response from the patient to the stimulation. In one example, the plurality of selectable duty cycles are further dependent on the at least one indication of the response. In one example, the at least one indication of the response comprises one or more of a perception threshold, a pain score, a pain coverage metric, or a measured neural response to the stimulation.

A method is disclosed for controlling an implantable stimulator device of a patient using an external device in communication with the implantable stimulator device. The method may comprise: receiving at a graphical user interface (GUI) of the external device a selection of a set of stimulation parameters for the implantable stimulator device to execute to provide stimulation to the patient; determining duration parameters comprising a minimum on duration, a maximum on duration, a minimum off duration, and a maximum off duration, wherein at least one of the duration parameters is dependent on the selected set of stimulation parameters; and providing on the GUI a first interface to select a duty cycle for the stimulation from a plurality of duty cycles, wherein each duty cycle selectable in the first interface selects both an on duration bounded between the minimum and maximum on duration and an off duration bounded between the minimum and maximum off duration.

In one example, the selected set of stimulation parameters defines the stimulation as a sequence of stimulation pulses. In one example, the selected set of stimulation parameters comprises a frequency of the stimulation pulses. In one example, the at least one of the duration parameters is dependent on the frequency of the selected set of stimulation parameters. In one example, the plurality of selectable duty cycles have values that decrease as the frequency of the selected set of stimulation parameters increases. In one example, the method further comprises receiving at the GUI at least one indication of a response from the patient to the stimulation. In one example, the plurality of selectable duty cycles are further dependent on the at least one indication of the response. In one example, the at least one indication of the response comprises one or more of a perception threshold, a pain score, a pain coverage metric, or a measured neural response to the stimulation. In one example, the first interface is configured to allow a user to step though the plurality of duty cycles from a minimum duty cycle to a maximum duty cycle. In one example, all of the duration parameters are dependent on the selected set of stimulation parameters. In one example, the selection of the set of stimulation parameters is received at a second interface provided on the GUI. In one example, the second interface is configured to allow a user to step though a plurality of sets of stimulation parameters to select the set of stimulation parameters. In one example, each of the plurality of sets of stimulation parameters comprises a frequency, an amplitude, and a pulse width of the stimulation. In one example, the plurality of sets of stimulation parameters are derived from a model for the patient determined from providing test stimulation to the patient. In one example, the method further comprises providing the test stimulation to the patient to determine the model. In one example, each of the sets of stimulation parameters comprises a charge rate, wherein the second interface is configured to step through the plurality of sets of stimulation parameters from a minimum charge rate to a maximum charge rate. In one example, the minimum charge rate and the maximum charge rate are expressed in microCoulombs per second, and respectively comprise 2.5 and 20, or 8 and 80, or 12 and 140, or 15 or 200, or 18.9 and 294, or 18.8 and 346, or 20 and 450. In one example, the minimum charge rate and the maximum charge comprise 2.5 and 20 if a frequency of the set of stimulation parameters comprises 10 Hz, or 8 and 80 if a frequency of the set of stimulation parameters comprises 50 Hz, or 12 and 140 if a frequency of the set of stimulation parameters comprises 100 Hz, or 15 or 200 if a frequency of the set of stimulation parameters comprises 200 Hz, or 18.9 and 294 if a frequency of the set of stimulation parameters comprises 600 Hz, or 18.8 and 346 if a frequency of the set of stimulation parameters comprises 10 Hz, or 20 and 450 if a frequency of if a frequency of set of stimulation parameters comprises 10 Hz set of stimulation parameters comprises 1000 Hz. In one example, the stimulation comprises sub-perception stimulation.

An external device is disclosed for controlling an implantable stimulator device of a patient, which may comprise: a graphical user interface (GUI) configured to allow a user to select a set of stimulation parameters for the implantable stimulator device to execute to provide stimulation to the patient; control circuitry configured to determine duration parameters comprising a minimum on duration, a maximum on duration, a minimum off duration, and a maximum off duration, wherein at least one of the duration parameters is dependent on the selected set of stimulation parameters; and wherein the GUI comprises a first interface configured to allow the user to select a duty cycle for the stimulation from a plurality of duty cycles, wherein each duty cycle selectable in the first interface selects both an on duration bounded between the minimum and maximum on duration and an off duration bounded between the minimum and maximum off duration.

In one example, the selected set of stimulation parameters defines the stimulation as a sequence of stimulation pulses. In one example, the selected set of stimulation parameters comprises a frequency of the stimulation pulses. In one example, the at least one of the duration parameters is dependent on the frequency of the selected set of stimulation parameters. In one example, the plurality of selectable duty cycles have values that decrease as the frequency of the selected set of stimulation parameters increases. In one example, the GUI is further configured to receive at least one indication of a response from the patient to the stimulation. In one example, the at least one the duration parameters is further dependent on the at least one indication of the response. In one example, the at least one indication of the response comprises one or more of a perception threshold, a pain score, a pain coverage metric, or a measured neural response to the stimulation. In one example, the first interface is configured to allow a user to step though the plurality of duty cycles from a minimum duty cycle to a maximum duty cycle. In one example, all of the duration parameters are dependent on the selected set of stimulation parameters. In one example, the GUI further comprises a second interface configured to allow the user to select the set of stimulation parameters. In one example, the second interface is configured to allow a user to step though a plurality of sets of stimulation parameters to select the set of stimulation parameters. In one example, each of the plurality of sets of stimulation parameters comprises a frequency, an amplitude, and a pulse width of the stimulation. In one example, the plurality of sets of stimulation parameters are derived by the control circuitry from a model stored in the external device. In one example, each of the sets of stimulation parameters comprises a charge rate, wherein the second interface is configured to step through the plurality of sets of stimulation parameters from a minimum charge rate to a maximum charge rate. In one example, the minimum charge rate and the maximum charge rate are expressed in microCoulombs per second, and respectively comprise 2.5 and 20, or 8 and 80, or 12 and 140, or 15 or 200, or 18.9 and 294, or 18.8 and 346, or 20 and 450. In one example, the minimum charge rate and the maximum charge comprise 2.5 and 20 if a frequency of the set of stimulation parameters comprises 10 Hz, or 8 and 80 if a frequency of the set of stimulation parameters comprises 50 Hz, or 12 and 140 if a frequency of the set of stimulation parameters comprises 100 Hz, or 15 or 200 if a frequency of the set of stimulation parameters comprises 200 Hz, or 18.9 and 294 if a frequency of the set of stimulation parameters comprises 600 Hz, or 18.8 and 346 if a frequency of the set of stimulation parameters comprises 10 Hz, or 20 and 450 if a frequency of if a frequency of set of stimulation parameters comprises 10 Hz set of stimulation parameters comprises 1000 Hz. In one example, the plurality of sets of stimulation parameters are configured to provide sub-perception stimulation for the patient.

A method is disclosed for controlling an implantable stimulator device of a patient using an external device in communication with the implantable stimulator device. The method may comprise: providing on the GUI of the external device an interface to select stimulation for the implantable stimulator device to provide to the patient, wherein the interface is configured to allow a user to step though a number of steps, wherein each step selects both: a duty cycle for the stimulation comprising an on duration and an off duration at which the stimulation will be repeatedly enabled and disabled, and a set of stimulation parameters for the stimulation.

In one example, the selected set of stimulation parameters defines the stimulation as a sequence of stimulation pulses. In one example, the selected set of stimulation parameters comprises a frequency of the stimulation pulses. In one example, the selected set of stimulation parameters further comprises an amplitude and a pulse width of the stimulation pulses. In one example, the selected duty cycle at each step is dependent on the frequency of the selected set of stimulation parameters. In one example, the selected duty cycle decreases as the frequency of the selected set of stimulation parameters increases. In one example, the interface is configured to step through the steps from a minimum duty cycle to a maximum duty cycle. In one example, the minimum duty cycle is defined by a minimum charge rate, and wherein the maximum duty cycle is defined by a maximum charge rate. In one example, the minimum charge rate and the maximum charge rate are expressed in microCoulombs per second, and respectively comprise 2.5 and 20, or 8 and 80, or 12 and 140, or 15 or 200, or 18.9 and 294, or 18.8 and 346, or 20 and 450. In one example, the minimum charge rate and the maximum charge comprise 2.5 and 20 if a frequency of the set of stimulation parameters comprises 10 Hz, or 8 and 80 if a frequency of the set of stimulation parameters comprises 50 Hz, or 12 and 140 if a frequency of the set of stimulation parameters comprises 100 Hz, or 15 or 200 if a frequency of the set of stimulation parameters comprises 200 Hz, or 18.9 and 294 if a frequency of the set of stimulation parameters comprises 600 Hz, or 18.8 and 346 if a frequency of the set of stimulation parameters comprises 10 Hz, or 20 and 450 if a frequency of if a frequency of set of stimulation parameters comprises 10 Hz set of stimulation parameters comprises 1000 Hz. In one example, the duty cycles at each step are defined using duration parameters including a minimum on duration, a maximum on duration, a minimum off duration, and a maximum off duration. In one example, the duty cycles at each step are defined using different combinations of on durations bounded between the minimum and maximum on durations, and off durations bounded between the minimum and maximum off durations. In one example, the sets of stimulation parameters at each step are derived from a model for the patient determined from providing test stimulation to the patient. In one example, the method further comprises providing the test stimulation to the patient to determine the model. In one example, each step comprises a charge rate, wherein the interface is configured to allow a user to step though the number of steps from a minimum charge rate to a maximum charge rate. In one example, the method further comprises receiving at the GUI at least one indication of a response from the patient to the stimulation. In one example, either of both of the duty cycle and the set of stimulation parameters at each step are dependent on the at least one indication of the response. In one example, the at least one indication of the response comprises one or more of a perception threshold, a pain score, a pain coverage metric, or a measured neural response to the stimulation. In one example, the stimulation comprises sub-perception stimulation.

An external device is disclosed for controlling an implantable stimulator device of a patient, which may comprise: a graphical user interface (GUI) configured to allow a user to select stimulation for the implantable stimulator device to provide to the patient, wherein the interface is configured to allow a user to step though a number of steps, wherein each step is configured to select both: a duty cycle for the stimulation comprising an on duration and an off duration at which the stimulation will be repeatedly enabled and disabled, and a set of stimulation parameters for the stimulation.

In one example, the selected set of stimulation parameters defines the stimulation as a sequence of stimulation pulses. In one example, the selected set of stimulation parameters comprises a frequency of the stimulation pulses. In one example, the selected set of stimulation parameters further comprises an amplitude and a pulse width of the stimulation pulses. In one example, the selected duty cycle at each step is dependent on the frequency of the selected set of stimulation parameters. In one example, the selected duty cycle decreases as the frequency of the selected set of stimulation parameters increases. In one example, the interface is configured to step through the steps from a minimum duty cycle to a maximum duty cycle. In one example, the minimum duty cycle is defined by a minimum charge rate, and wherein the maximum duty cycle is defined by a maximum charge rate. In one example, the minimum charge rate and the maximum charge rate are expressed in microCoulombs per second, and respectively comprise 2.5 and 20, or 8 and 80, or 12 and 140, or 15 or 200, or 18.9 and 294, or 18.8 and 346, or 20 and 450. In one example, the minimum charge rate and the maximum charge comprise 2.5 and 20 if a frequency of the set of stimulation parameters comprises 10 Hz, or 8 and 80 if a frequency of the set of stimulation parameters comprises 50 Hz, or 12 and 140 if a frequency of the set of stimulation parameters comprises 100 Hz, or 15 or 200 if a frequency of the set of stimulation parameters comprises 200 Hz, or 18.9 and 294 if a frequency of the set of stimulation parameters comprises 600 Hz, or 18.8 and 346 if a frequency of the set of stimulation parameters comprises 10 Hz, or 20 and 450 if a frequency of if a frequency of set of stimulation parameters comprises 10 Hz set of stimulation parameters comprises 1000 Hz. In one example, the duty cycles at each step are defined using duration parameters including a minimum on duration, a maximum on duration, a minimum off duration, and a maximum off duration. In one example, the duty cycles at each step are defined using different combinations of on durations bounded between the minimum and maximum on durations, and off durations bounded between the minimum and maximum off durations. In one example, the sets of stimulation parameters at each step are derived from a model for the patient stored in the external device. In one example, each step comprises a charge rate, wherein the interface is configured to allow a user to step though the number of steps from a minimum charge rate to a maximum charge rate. In one example, the GUI is further configured to receive at least one indication of a response from the patient to the stimulation. In one example, either of both of the duty cycle and the set of stimulation parameters at each step are dependent on the at least one indication of the response. In one example, the at least one indication of the response comprises one or more of a perception threshold, a pain score, a pain coverage metric, or a measured neural response to the stimulation. In one example, the stimulation comprises sub-perception stimulation.

### BRIEF DESCRIPTION OF THE DRAWINGS

FIG. 1 shows an Implantable Pulse Generator (IPG) useable for Spinal Cord Stimulation (SCS), in accordance with the prior art.

FIG. 2 shows an example of stimulation pulses producible by the IPG, in accordance with the prior art.

FIG. 3 shows stimulation circuitry useable in the IPG to independently set the current at each of the electrodes.

FIG. 4 shows various external systems capable of communicating with and programming stimulation in an IPG, in accordance with the prior art.

FIG. 5 shows a Graphical User Interface (GUI) of a clinician programmer external device for setting or adjusting stimulation parameters, in accordance with the prior art.

FIG. 6 shows a model derived from patients showing a surface denoting optimal sub-perception values for frequency and pulse width, and further including the patients' perception threshold pth as measured at those frequencies and pulse widths.

FIG. 7 shows the perception threshold pth plotted versus pulse width for a number of patients, and shows how results can be curve fit.

FIG. 8 shows a graph of parameter Z versus pulse width for patients, where Z comprises an optimal amplitude A for patients expressed as a percentage of perception threshold pth (i.e., Z=A/pth).

FIGS. 9A-9F show an algorithm used to derive a range of optimal sub-perception stimulation parameters (e.g., F, PW, and A) for a patient using the modelling information of FIGS. 6-8, and using perception threshold measurements taken on the patient.

FIG. 10 shows use of the optimal stimulation parameters in a patient external controller, including a user interface that allows the patient to adjust stimulation within the range.

FIGS. 11A-11F show the effect of statistic variance in the modelling, leading to the result that determined optimal stimulation parameters for a patient may occupy a volume. User interfaces for the patient external controller are also shown to allow the patient to adjust stimulation within this volume.

FIGS. 12A and 12B show GUI aspects for allowing a user to navigate stimulation parameters sets within the optimal stimulation parameters and/or subsets.

FIGS. 13A-13C show use of a neural dose adjustment interface useable in the GUI for allowing a user to navigate stimulation parameters sets within the optimal stimulation parameters and/or subsets.

FIGS. 14A-15B show the addition of a duty cycling adjustment interface useable in the GUI for allowing a user to navigate duty cycle on and off times for the optimal stimulation parameters and/or subsets.

FIGS. 16A-16B show a modification to operation of the duty cycling adjustment interface.

FIG. 17 shows use of a combined neural dose adjustment and duty cycling adjustment interface for allowing a user to simultaneously navigate stimulation parameters and duty cycles.

FIG. 18 shows further details of the GUI that includes the neural dose adjustment and duty cycling adjustment interfaces.

FIG. 19 shows a modification in which one or more metrics indicative of a patent's response to stimulation is used to modify the determination of duty cycles for the patient.

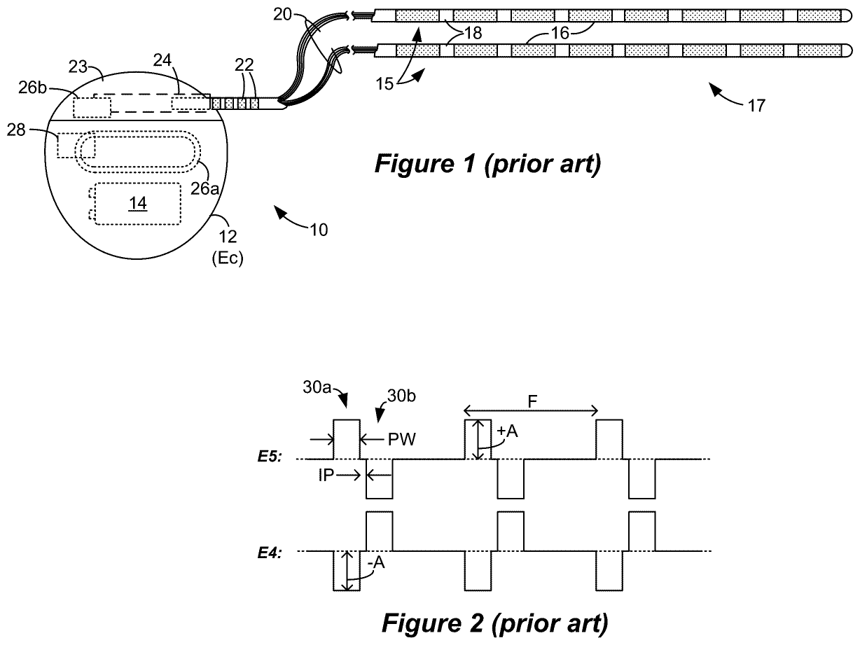

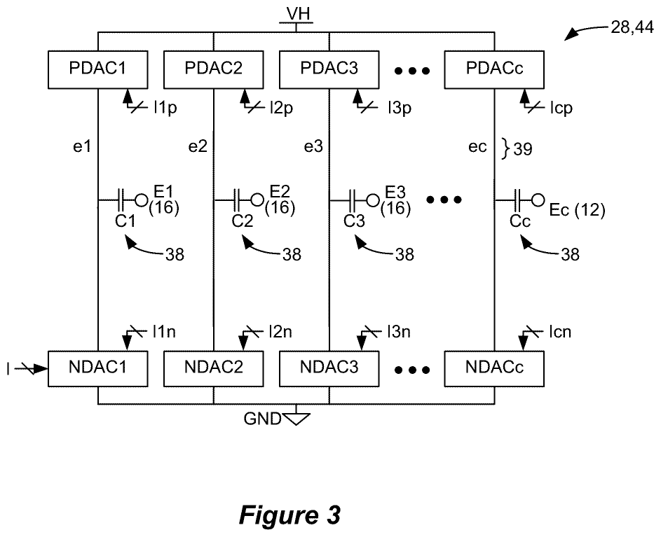

### DETAILED DESCRIPTION

While Spinal Cord Stimulation (SCS) therapy can be an effective means of alleviating a patient's pain, such stimulation can also cause paresthesia. Paresthesia—sometimes referred to a “supra-perception” therapy—is a sensation such as tingling, prickling, heat, cold, etc. that can accompany SCS therapy. Generally, the effects of paresthesia are mild, or at least are not overly concerning to a patient. Moreover, paresthesia is generally a reasonable tradeoff for a patient whose chronic pain has now been brought under control by SCS therapy. Some patients even find paresthesia comfortable and soothing.

Nonetheless, at least for some patients, SCS therapy would ideally provide complete pain relief without paresthesia—what is often referred to as “sub-perception” or sub-threshold therapy that a patient cannot feel. Effective sub-perception therapy may provide pain relief without paresthesia by issuing stimulation pulses at higher frequencies. Unfortunately, such higher-frequency stimulation may require more power, which tends to drain the battery 14 of the IPG 10. See, e.g., U.S. Patent Application Publication 2016/0367822. If an IPG's battery 14 is a primary cell and not rechargeable, high-frequency stimulation means that the IPG 10 will need to be replaced more quickly. Alternatively, if an IPG battery 14 is rechargeable, the IPG 10 will need to be charged more frequently, or for longer periods of time. Either way, the patient is inconvenienced.

In an SCS application, it is desirable to determine a stimulation program that will be effective for each patient. A significant part of determining an effective stimulation program is to determine a “sweet spot” for stimulation in each patient, i.e., to select a position for stimulation in the electrode array 17 by selecting which electrodes should be active (E) and with what polarities (P) and relative amplitudes (X %). Preferably, this position treats a neural site at which pain originates in a patient. Selecting electrodes proximate to this neural site of pain can be difficult to determine, and experimentation is typically undertaken to select the best combination of electrodes to provide a patient's therapy.

As described in U.S. Pat. No. 10,576,282, which is incorporated by reference it its entirety, selecting electrodes for a given patient can be even more difficult when sub-perception therapy is used, because the patient does not feel the stimulation, and therefore it can be difficult for the patient to feel whether the stimulation is “covering” his pain and therefore whether selected electrodes are effective. Further, sub-perception stimulation therapy may require a “wash in” period before it can become effective. A wash in period can take up to a day or more, and therefore sub-perception stimulation may not be immediately effective, making electrode selection more difficult. The '282 Patent describes a technique whereby sweet spot searching is accomplished using supra-perception stimulation that a patient can feel. This helps in targeting the neural site requiring stimulation to alleviate the patient's symptoms. Once this sweet spot has been determined—i.e., once the position of stimulation in the electrode array 17 has been determined for the patient—the stimulation can be adjusted to sub-perception levels to good effect and with faster wash in times.

Once the location of stimulation in the electrode array has been determined for the patient, it is typically desired to determine values for the stimulation parameters at that location, such as the frequency of the pulses (F), the amplitude of the pulses (A, whether current or voltage), and the pulse width (PW) of those pulses. In this regard, the '282 Patent also discloses that statistically significant correlations exist between pulse width (PW) and frequency (F) where an SCS patient will experience a reduction in back pain without paresthesia (sub-perception). Use of this information can be helpful in deciding what pulse width is likely optimal for a given SCS patient based on a particular frequency, and in deciding what frequency is likely optimal for a given SCS patient based on a particular pulse width. Beneficially, this information suggests that paresthesia-free sub-perception SCS stimulation can occur at frequencies of 10 kHz and below, and more specifically at 1 kHz or below (and as low as 2 Hz). Use of such low frequencies allows sub-perception therapy to be used with much lower power consumption in the patient's IPG.

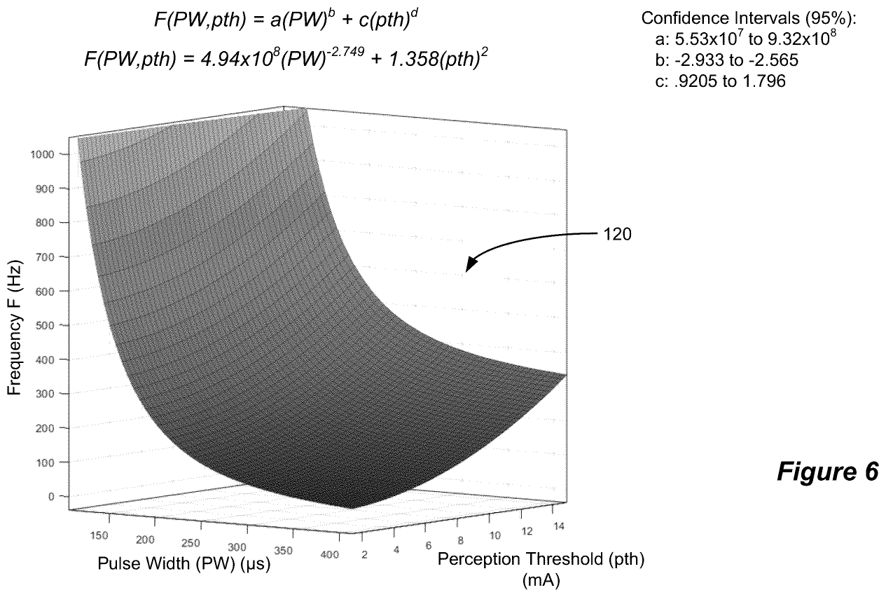

U.S. Patent Application Publication 2020/0009367, which is also incorporated herein by reference in its entirety, builds upon the learnings disclosed in the above-referenced '282 patent. The '367 Publication provides testing and modelling to arrive at optimal sub-perception stimulation parameters for a patient, and this technique is summarized here in FIGS. 6-11F. These figures disclose an approach to optimal sub-perception modelling that takes into account a patient's perception threshold (pth). Perception threshold, pth, comprises a lowest magnitude at which the patient can feel the effects of paresthesia (e.g., in mA), with magnitudes below this causing sub-perception stimulation. Different patients will have different perception thresholds, and this results in significant part because the distance of the electrode array 17 to spinal neural fibers can differ in various patients. If the electrode array is closer to the spinal cord, a patient will typically experience perception at lower magnitudes, i.e., pth will be lower for such patients. If the electrode array is farther from the spinal cord in a patient, the perception threshold pth will be higher. When modelling optimal stimulation parameters for a patient is significant because it allows optimal sub-perception amplitudes A to be determined for the patient in addition to optimal frequencies and pulse widths.

FIG. 6 shows a model 120 developed based on testing a number of patients, which shows frequencies and pulse widths they found optimal, as well as the patients' perception threshold at those frequencies and pulse widths. FIG. 6 shows mean values as determined by three-dimensional regression fitting, which yields model 120 as a surface in Frequency-Pulse Width-Perception Threshold space. Data as represented in FIG. 6 was taken at frequencies of 1 kHz and below. Data at these frequencies is of particular interest, because, as already mentioned, lower frequencies are more considerate of energy usage in an IPG, and hence is it particularly desirable to prove the utility of sub-perception stimulation in this frequency range. As can be seen by the equation in FIG. 6, data taken from the patients was modelled with a good fit by assuming that frequency varies with both pulse width (a(PW)^{b}) and perception threshold pth (c(pth)^{d}) in accordance with power functions. While these functions provided suitable fitting, other types of mathematical equations could be used for fitting as well. Note that frequency, pulse width, and perception threshold are not simply proportionally related or inversely proportionately related model 120, but are instead related by non-linear functions.

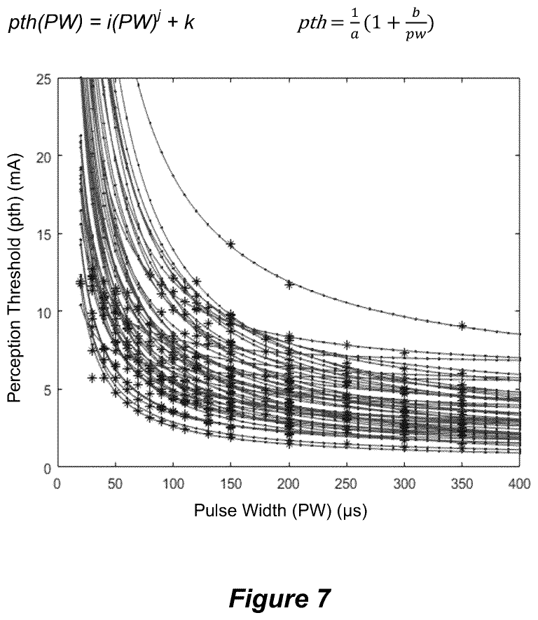

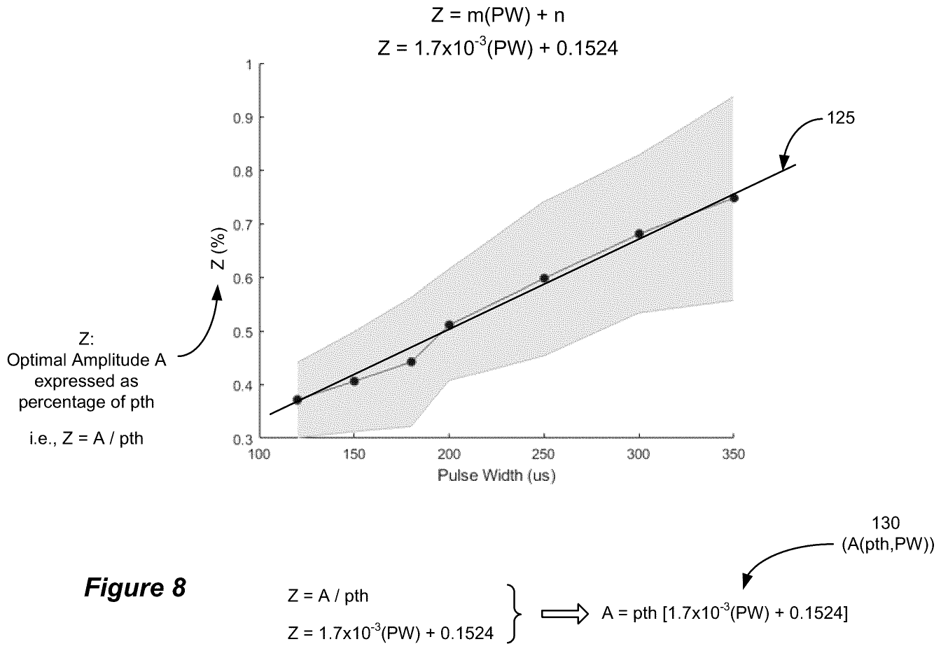

FIG. 7 shows further observations noticed from tested patients, and provides another modelling aspect that along with model 120 can be used to determine optimal sub-threshold stimulation parameters for a patient. FIG. 7 shows how perception threshold pth varies as a function of pulse width for the tested patients, with each patient being represented by a different line in the graph of FIG. 7. Analysis of each of the lines suggests that the relationship between pth and PW can be well modeled with a power function, i.e., pth(PW)=i(PW)^{j}+k, although again other mathematic functions could be used for fitting as well, such as a polynomial function, an exponential function, etc. For example, a Weiss-Lapicque, or strength-duration, equation can also be used, which relates the amplitude and pulse width required to attain a threshold. The equation takes the form pth=(1/a)(1+b/PW), and when data for different patients are averaged, constants a=0.60 and b=317 result with good fitting results, where these values represent the mean constant parameters extracted from the population data. The data of FIG. 7 was taken for each patient at a nominal frequency such as 200 to 500 Hz, with further analysis confirming that the results do not vary considerably with frequency (at least at frequencies of 150 Hz and higher, using biphasic pulses with active recharge). Pulse widths in FIG. 7 were limited to the range of approximately 100 to 400 microseconds because pulse widths in this range were shown to have unique sub-perception therapeutic effectiveness at frequencies of 1 kHz and lower in the above-referenced '282 patent.

FIG. 8 shows still further observations noted from the tested patients, and provides yet another modeling aspect. FIG. 8 shows how optimal sub-perception amplitudes A for patients vary in accordance with a patients' perception thresholds pth, as well as with pulse width. In the graph in FIG. 8, the vertical axis plots a parameter Z, which relates a patients' perception thresholds pth and their optimal amplitudes A (which in a sub-perception therapy would be lower than pth). Specifically, Z is the optimal amplitude expressed as a percentage of pth, i.e., Z=A/pth. As FIG. 8 shows, Z varies with pulse width. At smaller pulse widths (e.g., 150 microsecond), Z is relatively low, meaning that the optimal amplitude A for patients was noted to be considerably lower than their perception thresholds (e.g., A=40% of pth). At longer pulse widths (e.g., 350 microseconds), Z is higher, meaning that the optimal amplitude A for patients was noted to be closer to their perception thresholds (e.g., A=70% of pth). Z and PW as noted from testing various patients generally have a linear relationship over the pulse widths tested, and so linear regression was used to determine their relationship, yielding Z=0.0017(PW)+0.1524 (125). Again, testing in FIG. 8 was limited to the general range of 100 to 400 microseconds noted to be useful for sub-perception therapy at less than 1 kHz. Because Z varies with pulse width, and because Z also varies with optimal amplitude A and perception threshold pth (Z=A/pth), the modelling of FIG. 8 allows optimal amplitude A to be modelled as a function of both perception threshold pth and pulse width PW, i.e., A=pth [0.0017(PW)+0.1524] (130).

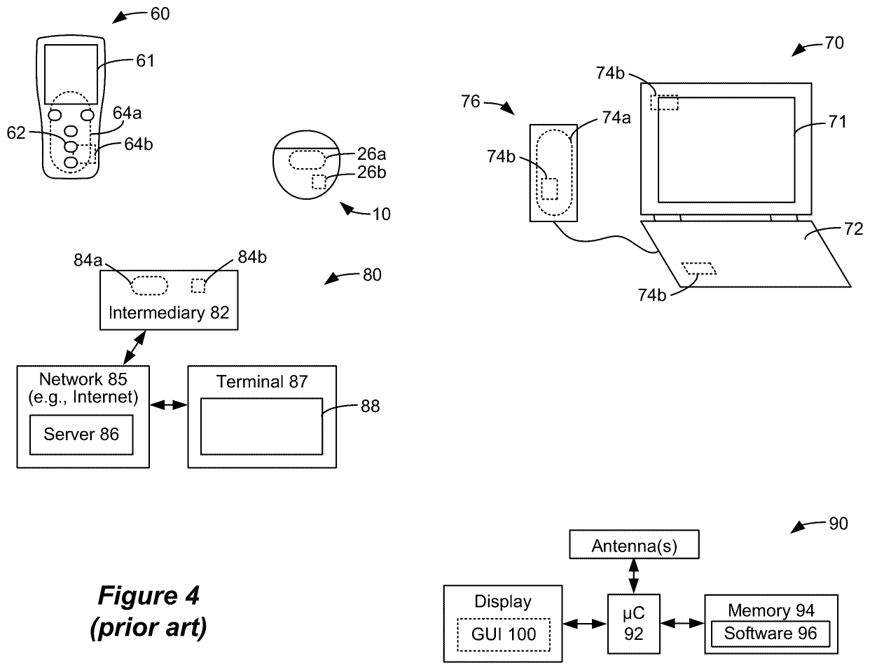

From these observations, the '367 Publication discloses an algorithm 150 that can be used to provide personalized sub-perception therapy for particular patients. This algorithm 150 can be implemented on an external system such as a clinician programmer 70 (FIG. 4), and results in the determination of a range or volume of optimal sub-perception parameters (e.g., F, PW, and A) for the patient. Preferably, as last step in the algorithm 150, the range or volume of optimal sub-perception parameters is transmitted to the patient' s external controller 60 to allow the patient to adjust their sub-perception therapy within this range or volume.

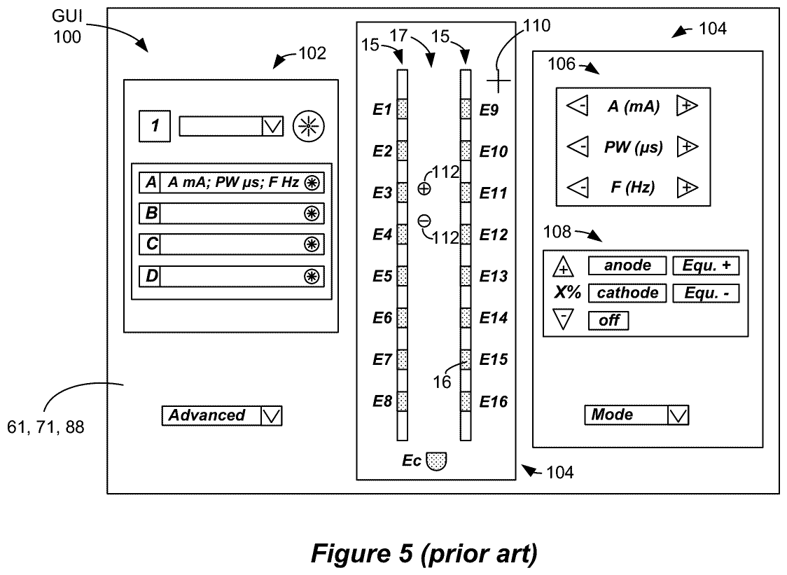

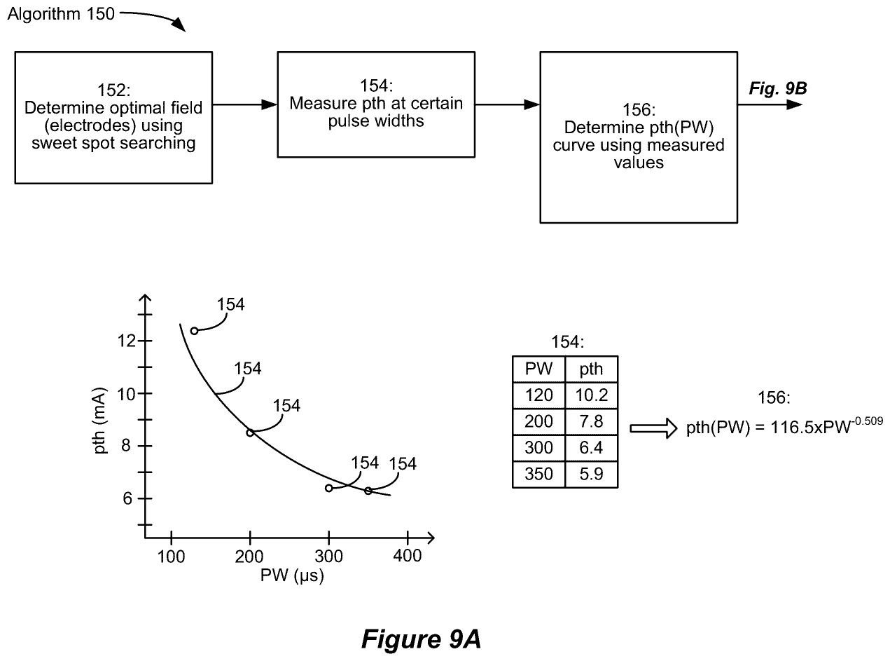

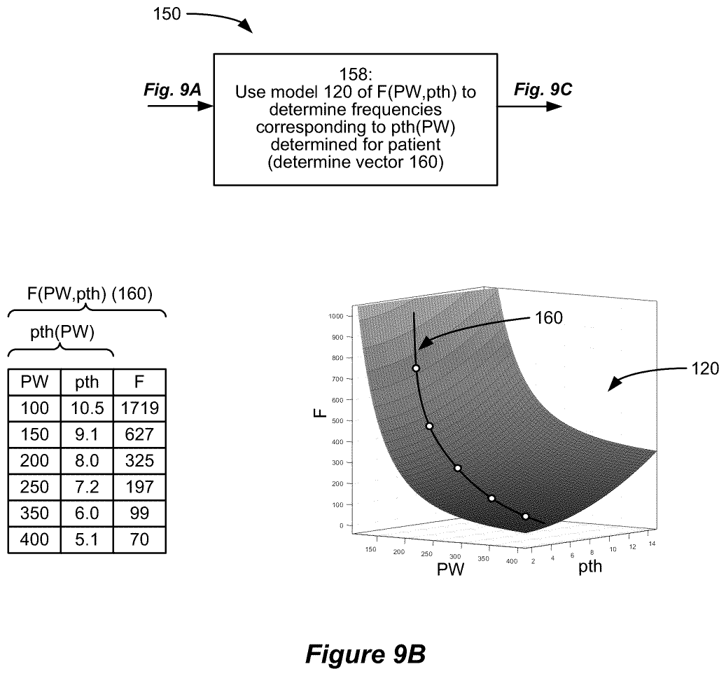

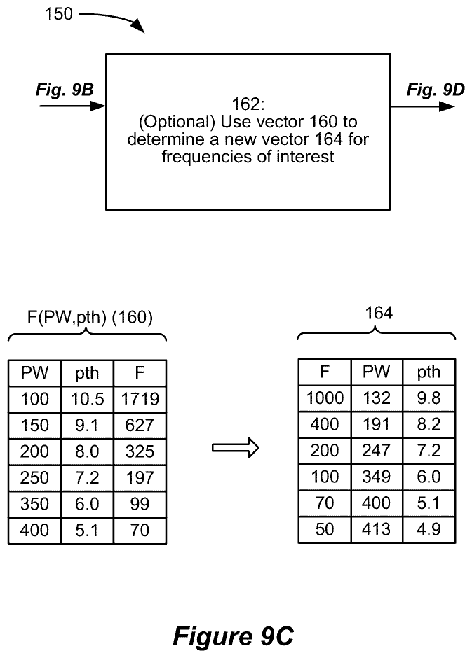

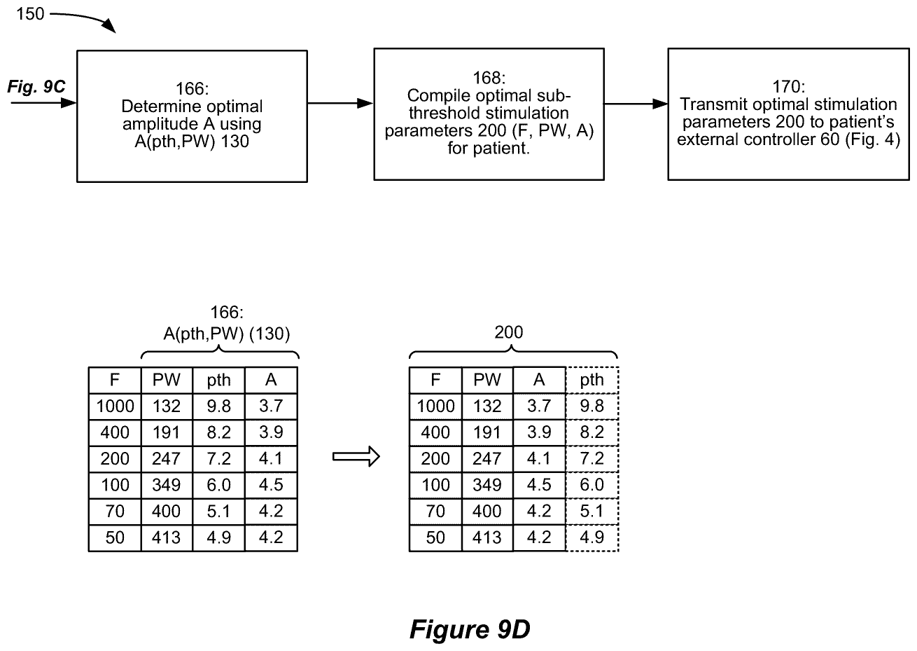

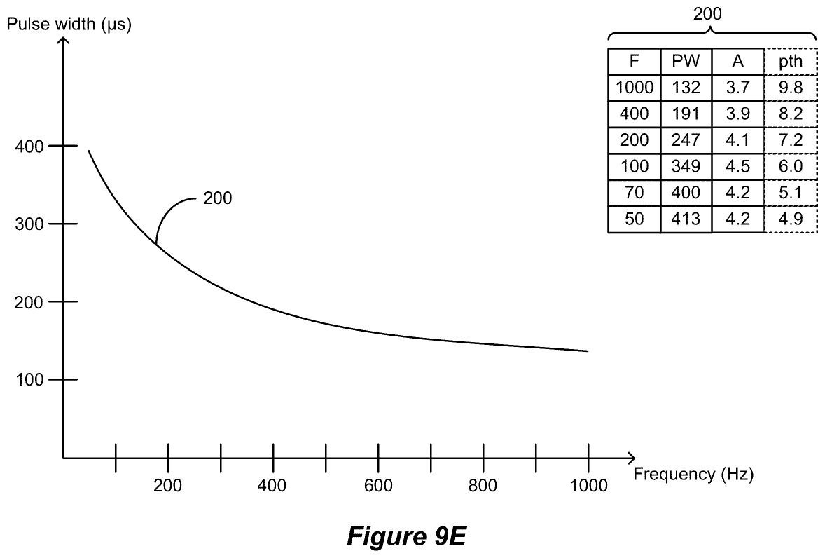

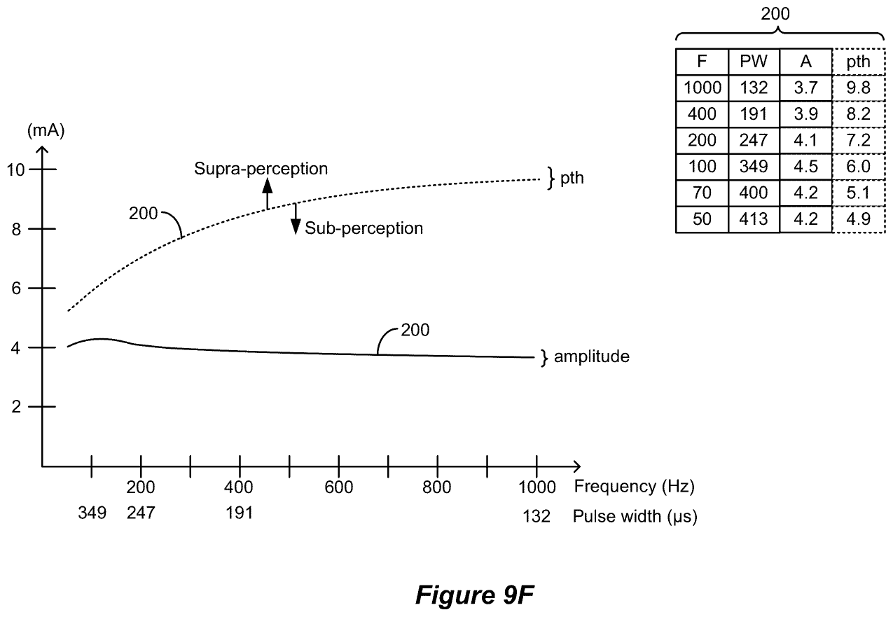

The algorithm 150, shown starting in FIG. 9A, starts in step 152 by determining for a given patient the sweet spot in the electrode array at which therapy should be applied—i.e., by identifying which electrodes should be active and with what polarities and percentages (X %), which as noted earlier determines the location of the stimulation in the electrode array 17. The results of sweet spot searching may already be known for a given patient, and thus step 152 should be understood as optional. Step 152 can be accomplished by moving the poles 112 (FIG. 5) in the electrode array, as well as by adjust other stimulation parameters, as discussed in detail in the '282 patent for example.

At step 154, the patient's perception threshold pth is measured at various pulse widths by providing test pulses at the sweet spot determined earlier. Testing of different pulse widths can occur at a nominal frequency such as in the range of 200 to 500 Hz. Determining pth at each given pulse width involves applying the pulse width, and gradually increasing the amplitude A to a point where the patient reports feeling the stimulation (paresthesia), resulting in a pth expressed in terms of amplitude (e.g., milliamps). Alternatively, determining pth at each given pulse width can involve decreasing the amplitude A to a point where the patient reports no longer feels the stimulation (sub-perception). Testing at step 154 of a particular patient is shown graphically and in tabular form in FIG. 9A. Here, it is assumed that the patient in question has a paresthesia threshold pth of 10.2 mA at a pulse width of 120 microseconds; a pth of 5.9 mA at a pulse width of 350 microseconds, and other values between these.

Next, in step 156, the algorithm 150 in the external system (e.g., the clinician programmer 70) models the pth v. PW data points measured in step 156, and curve fits them to a mathematical function. This mathematical function could be one noticed earlier to well model pth and PW in other patients, such as a power function or the Weiss Lapicque equation, as discussed earlier with respect to FIG. 7. The measured data in table 154, as well as the determined curve-fit relationship pth(PW) 156 determined for the patient may be stored in memory in the clinician programmer 70 for use in subsequent steps.

Next, and referring to FIG. 9B, algorithm 150 compares the pth(PW) relationship determined in step 156 to the model 120 (FIG. 6). This is explained with reference to a table shown in FIG. 9B. In this table, values for pth and PW are populated, as determined by the pth(PW) (156) determined in FIG. 9A. As can be seen, discrete pulse width values of interest (100 microseconds, 150 microseconds, etc.) may be used (which may vary from the exact pulse widths used during patient testing in step 154). While only six rows of PW ν.pth values are shown in the table of FIG. 9B, this could be a much longer vector of values, with pth determined at discrete PW steps (such as 10 microsecond steps).

The pth v. PW values are in step 158 compared against the three-dimensional model 120 to determine frequencies F that would be optimal at these various pth v. PW pairs. In other words, the pth and PW values are provided as variables into the surface fit equation (F(PW,pth)) 120 in FIG. 6 to determine optimal frequencies, which frequencies are also shown as populated into the chart in FIG. 9B. At this point, the table in FIG. 9B represents a vector 160 relating pulse widths and frequencies that are optimal for the patient, and that in addition includes the perception threshold for the patient at these pulse width and frequencies values. In other words, a vector 160 represents values within the model 120 that are optimal for the patient. Note that vector 160 for the patient can be represented as a curved line along the three-dimensional model 120, as shown in FIG. 9B.

Next, and as shown in step 162 in FIG. 9C, the vector 160 can optionally be used to form another vector 164, which contains values of interest or more practically values that may be supportable by the IPG. For example, notice that vector 160 for the patient includes frequencies at higher values (e.g., 1719 Hz), or otherwise at odd values (such as 627 and 197 Hz). Frequencies at higher values may not be desirable to use, because even if effective for the patient, such frequencies will involve excessive power draws. Moreover, the IPG at issue may only be able to provide pulses with frequencies at discrete intervals (such as in 10 Hz increments). Therefore, in vector 164, frequencies of interest or that are supported are chosen (e.g., 1000 Hz, 400 Hz, 200 Hz, 100 Hz, etc.), and then corresponding values for PW and pth are interpolated using vector 160. Although not shown, it may be useful to formulate vector 160 as an equation F(PW,pth)) to make vector 164 easier to populate. Nonetheless, vector 164 includes essentially the same information as vector 160, albeit at desirable frequencies. Realize that only certain pulse widths may be supportable by the IPG (e.g., in 10 microsecond increments). Therefore, the pulse widths in vector 164 may be adjusted (e.g., rounded) to nearest supported values, although this isn't shown in the drawings.

Next, and referring to FIG. 9D, the algorithm 150 in step 166 determines optimal amplitudes for the pulse width and pth values in vector 164 (or vector 160 if vector 164 isn't used). This occurs by using the amplitude function 130 determined earlier in FIG. 8, i.e., A(pth,PW). Using this function, an optimal amplitude A can be determined for each pth, PW pair in the table.

At this point, in step 168, optimal sub-threshold stimulation parameters F, PW, A 200 are determined as a model specific to the patient. Optimal stimulation parameters 200 may not need to include the perception threshold, pth: although pth was useful to determine optimal subthreshold amplitude A for the patient (step 166), it may no longer be a parameter of interest as it is not a parameter that the IPG produces. However, in other examples discussed later, it can be useful to include pth with the optimal parameters 200, as this can allow a patient to adjust their stimulation to a supra-perception level if desired. At this point, optimal stimulation parameters 200 may then be transmitted to the IPG for execution, or as shown in step 170, they may be transmitted to the patient's external controller 60, as described next.

FIGS. 9E and 9F depict optimal parameters 200 in graphical form. While optimal parameters 200 in this example comprise a three-dimensional range or line of coordinates (F, PW, and A), they are depicted in two two-dimensional graphs for easier illustration: FIG. 9E shows the relationship between frequency and pulse width, and FIG. 9F shows the relationship between frequency and amplitude. Note also that FIG. 9F shows the paresthesia threshold pth, and additionally shows on the X-axis the pulse width corresponding to the various frequencies from FIG. 9E. Note that the shapes of the data on these graphs could vary from patient to patient (e.g., based on the pth measurements of FIG. 9A), and could also change depending on the underlying modelling used (e.g., FIGS. 6-8). The various shapes of the trends shown thus should not be construed as limiting.

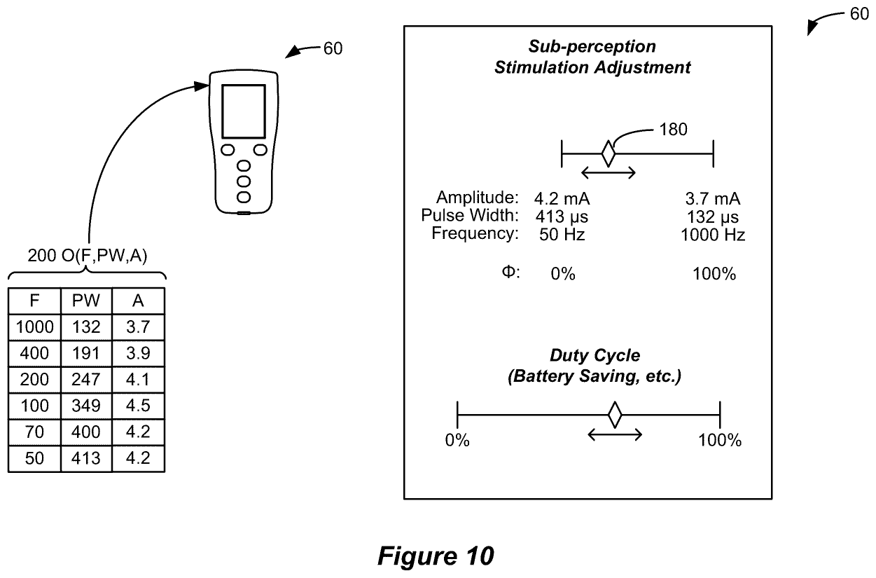

The optimal sub-perception stimulation parameters 200 determined by the algorithm 150 are shown in tabular form in FIG. 10. However, it should be understood these optimal parameters (0) may be curve fit using an equation that includes frequency, pulse, and amplitude (i.e., 0=f(F,PW,A)). Because each of these coordinates are optimal, it may be reasonable to allow the patient to use them with their IPG, and as a result the optimal parameters 200 may be sent from the clinician programmer 70 to the patient external controller 60 (FIG. 4) to allow the patient to select between them. In this regard, the optimal parameters 200, whether in tabular form or in the form of an equation, can be loaded into control circuitry of the external controller 60.

Once loaded, the patient can access a menu in the external controller 60 to adjust the therapy the IPG provides consistent the optimal parameters 200. For example, FIG. 10 shows a graphical user interface (GUI) of the external controller 60 as displayed on its screen. The GUI includes means to allow the patient to simultaneously adjust the stimulation within the range of determined optimal stimulation parameters 200. In one example, a slider is included in the GUI with a moveable cursor 180. The patient may select the cursor 180 and in this example move it to the left or right to adjust the frequency of stimulation pulses in their IPG. Moving it to the left reduces the frequency down to a minimum value included in the optimal parameters 200 (e.g., 50 Hz). Moving the cursor 180 to the right increases the frequency to a maximum value included in the optimal parameters (e.g., 1000 Hz). As the cursor 180 is moved and the frequency of stimulation is changed, the pulse widths and amplitudes can be simultaneously adjusted as reflected in optimal parameters 200. For example, at F=50 Hz, the amplitude is automatically set to A=4.2 mA, and the pulse width is set to 413 microseconds. At F=1000 Hz, the amplitude is set to A=3.7 mA, and the pulse width is set to 132 microseconds. In effect the cursor 180 allows the patient to navigate through the optimal parameters 200 to find a F/PW/A setting they prefer, or simply to choose stimulation parameters that are still effective but require lower power draws from the IPG (e.g., at lower frequencies). Note that the frequency, pulse width, and amplitude may not be adjusted proportionately or inversely proportional with respect to each other but will follow non-linear relationships in accordance with the underlying modelling.

In another example, it may be useful to allow the patient to adjust stimulation without knowledge of the stimulation parameters, i.e., without displaying the parameters, which may be too technical for the patient to understand. In this regard, the slider can be labeled with a more generic parameter, such as φ, which the patient can adjust, such as between 0 and 100%. The three-dimensional simulation parameters A, PW, and F can be mapped to this one-dimensional parameter φ (e.g., 4.2 mA, 413 μs, and 50 Hz can equal 0% as shown). Generally speaking, the patient may understand parameter φ as a sort of “intensity” or “neural dose” which is higher at higher percentages. This may in fact be true depending on the manner in which the optimal stimulation parameters 200 are mapped to φ.

It should be appreciated that while the GUI of the external controller 60 does allow the patient some flexibility to modify stimulation parameters for his IPG, it is also simple, and beneficially allows the patient to adjust all three stimulation parameters simultaneously using a single user interface element, all while being ensured that the resulting stimulation parameters will provide optimal sub-threshold stimulation.

Other stimulation adjustment controls may be provided by the external controller 60 as well. For example, as shown in FIG. 10, another slider can allow the patient to adjust the duty cycle to control the extent to which pulses will be continually running (100%) or completely off (0%). A duty cycle in the middle (e.g., 50%) will mean that pulses will run for a period of time (from second to minutes) and will then be off for that exact same duration. Because “duty cycle” may be a technical concept that a patient would not intuitively understand, note that the duty cycle may be labeled in a more intuitive manner. Thus, and as shown, the duty cycle adjustment may be labeled differently. For example, because lower duty cycles would affect lower power draws, the duty cycle slider may be label as a “battery saving” feature, as a “total energy” feature, a “total neural charge dose” feature, or the like, which may be easier for the patient to understand. Duty cycling may also comprise a feature in the external controller 60 that is locked to the patient, and only made accessible to a clinician for example, upon entering an appropriate password or other credentials. Note that the duty cycle could be smoothly adjusted, or made adjustable in pre-set logical increments, such as 0%, 10%, 20%, etc.

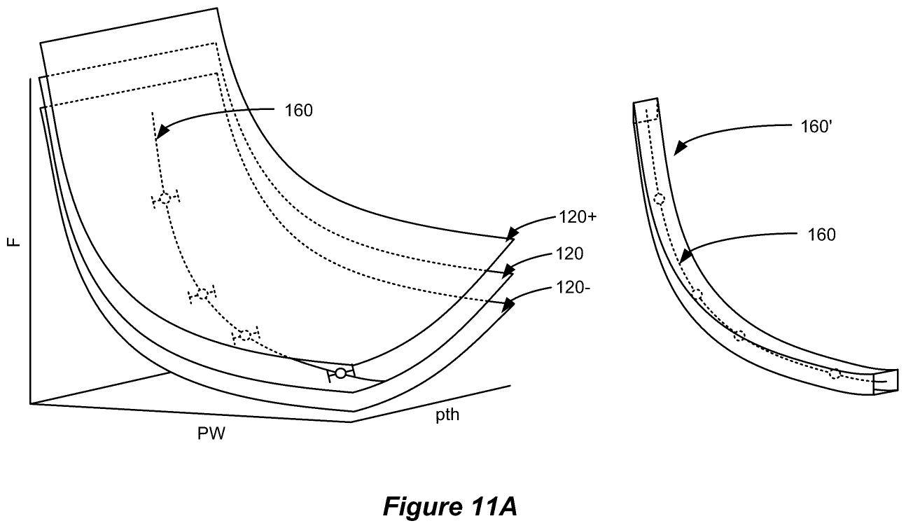

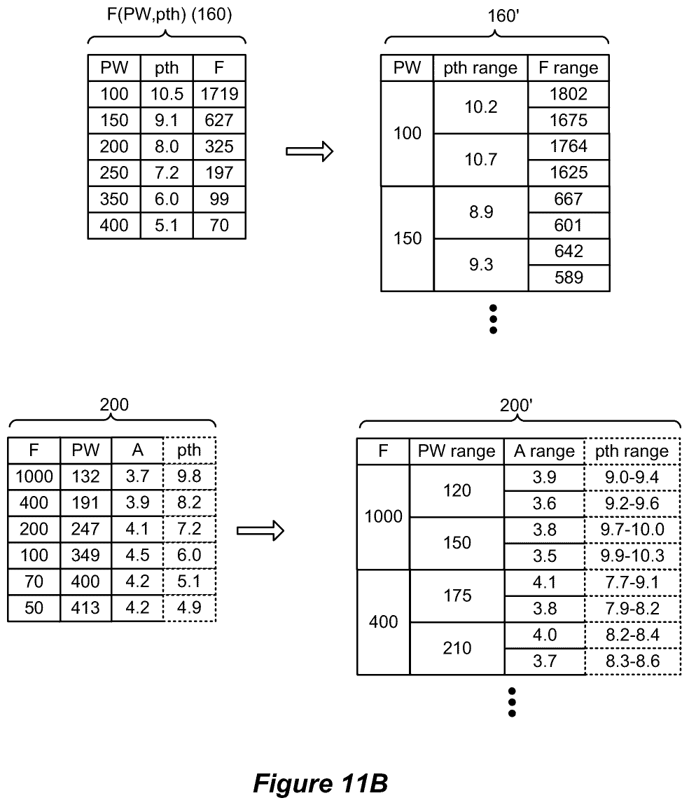

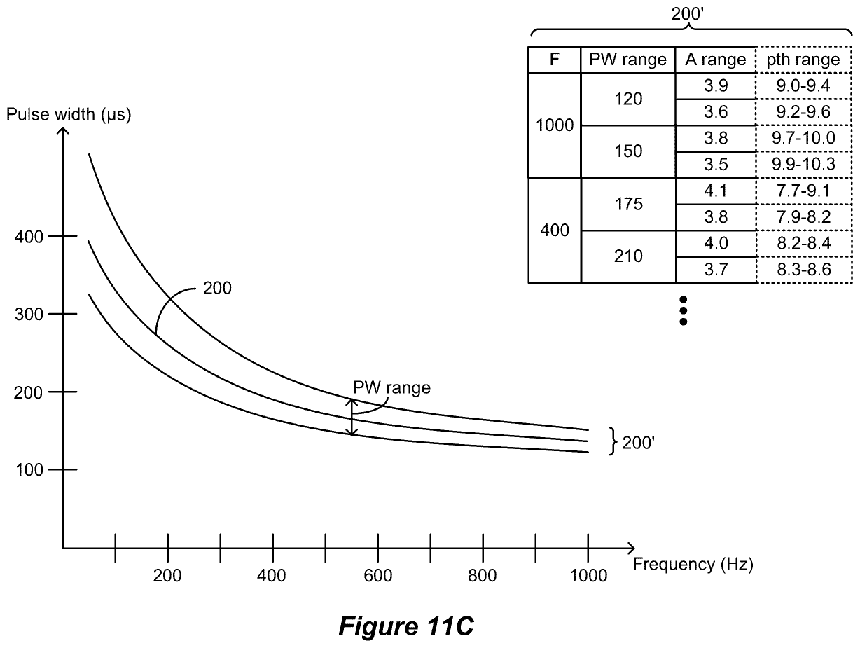

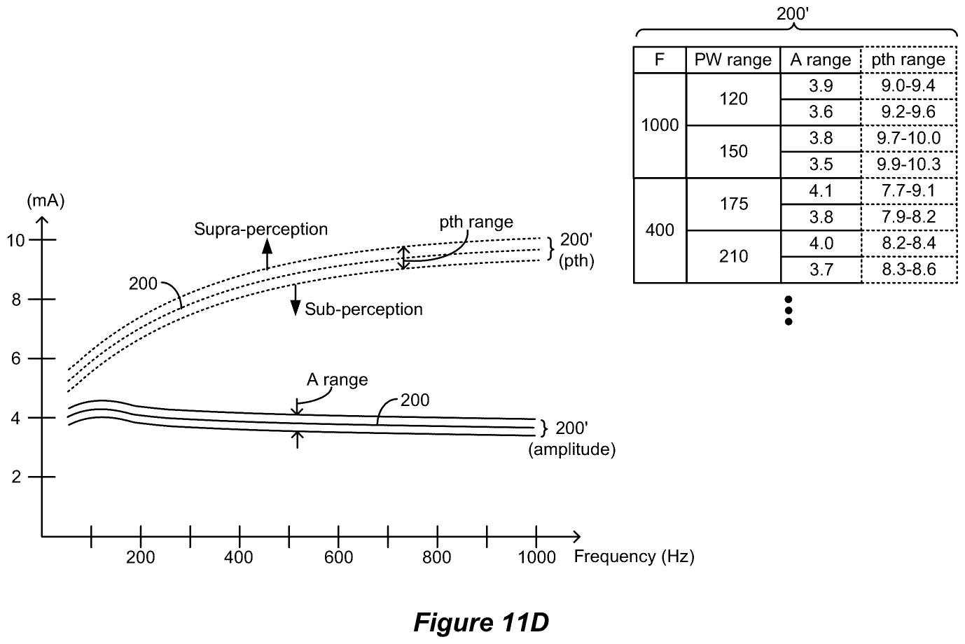

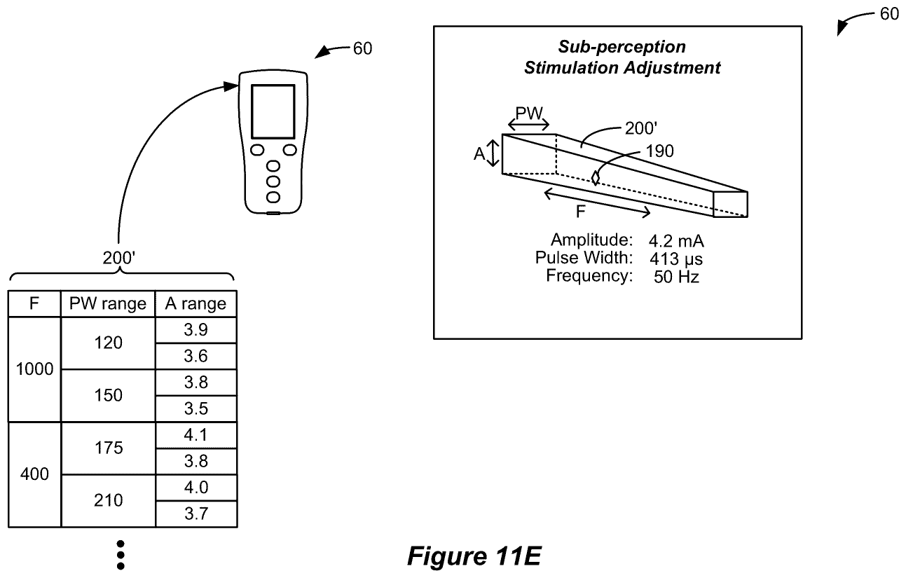

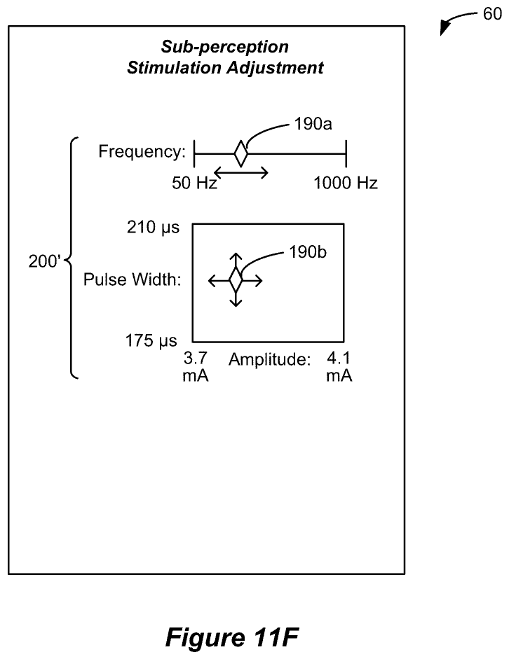

FIGS. 11A-11D address the practicality that the modeling leading to the determination of optimal parameters 200 may not be perfect. For example, model 120—modeling frequency as a function of PW and pth (F(PW,pth)); FIG. 6)—is averaged from various patients, and can have some statistical variance. This is illustrated simply in FIG. 11A by showing surfaces 120+ and 120− that are higher and lower from the mean as reflected in surface model 120. Surfaces 120+ and 120− may represent some degree of statistical variance or error measure, such as plus or minus one sigma, and may in effect generically comprise error bars beyond which the model 120 is no longer reliable. These error bars 120+ and 120− (which may not be constant over the entire surface 120) can also be determined from an understanding of the statistical variance in the various constants assumed during modeling. For example, values a, b, c, and d in model 120 may be determined with different measures of confidence. Likewise, values used to model the relationship of pth and pulse width (FIG. 7) may have different measure of confidence, as may values m and n used to model the relationship between optional amplitude, pth, and PW (FIG. 8). Over time, as data is taken on more patients, it would be expected that the confidence of these models would improve. In this regard, note that algorithm 150 can easily be updated with new modeling information from time to time by loading new modeling information into the clinician programmer 70.

Statistical variance means that optimal stimulation parameters determined by algorithm 150 may fall within a volume. This is illustrated in FIG. 11A as concerns the vector 160 determined for the patient (see FIG. 9B). Given statistical variance, vector 160 may comprise a rigid line within a volume 160'. In other words, there may not be a one-to-one correspondence of PW, pth, and F, as was the case for vector 160 in FIG. 9B. Instead, for any given variable (such as pulse width), the pth as determined for the patient (using the pth(PW) model in step 156) may vary in a range between statistically-significant maximum and minimum values, as shown in FIG. 11B. Statistical variation in model 120 (FIG. 6) may also mean that maximum and minimum frequencies may be determined for each maximum and minimum pth in step 158. As this trickles through the algorithm 150, the optimal stimulation parameters 200 may also not have one-to-one correspondence between frequency, pulse width, and amplitude. Instead, and as shown in FIG. 11B, for any frequency, there may be a range of optimal pulse widths and a range of optimal amplitudes A that are statistically significant and provide good therapeutic results for a patient, as shown by optimal stimulation parameters 200′. In other words, optimal stimulation parameters 200′ may be determined that define a statistically-significant volume of coordinates in Frequency-Pulse Width-Amplitude space rather than a line of coordinates. Paresthesia threshold pth may also vary within a range, and as noted earlier can be useful to include in the optimal stimulation parameters 200′, because pth may be helpful to permitting the patient to vary stimulation from sub-perception to supra-perception, as discussed in examples disclosed in the above-reference '367 Publication.

FIGS. 11C and 11D depict optimal parameters 200' in graphical form, showing at each frequency a statistically-relevant range of pulse widths, and a statistically-relevant range of amplitudes appropriate for the patient. While optimal parameters 200′ in this example comprise a three-dimensional volume of coordinates (F, PW, and A), they are depicted in two two-dimensional graphs for easier illustration, similar to what occurred earlier in FIGS. 9E and 9F: FIG. 11C shows the relationship between frequency and pulse width, and FIG. 11D shows the relationship between frequency and amplitude. Note also that FIG. 11D shows the paresthesia threshold pth, which like pulse width and amplitude can statistically vary within a range. Optimal stimulation parameters 200 (determined without statistical variance, see FIGS. 9E and 9F) are also shown for each of the parameters, and as expected fall within the broader volume for the parameters specified by 200′.

With a volume of optimal parameters 200′ defined, it may then be useful to allow the patient to use his external controller 60 to navigate different settings within this volume of optimal parameters 200′, which as before can involve transmitting the optimal parameter 200′ to the patient's external controller (see FIG. 9D, 170). This is shown in one example in FIG. 11E. Here, the GUI of the external controller 60 displays not a single linear slider, but a three-dimensional volume representative of the volume 200′ of optimal parameters, with different axes representing changes the patient can make in frequency, pulse width, and amplitude. As before, the GUI of the external controller 60 allows the patient some flexibility to modify stimulation parameters for his IPG, and allows the patient to adjust all three stimulation parameters simultaneously through one adjustment action and using a single user interface element.

Different GUIs to allow the patient to navigate through the determined volume of optimal parameters 200′ are possible, and FIG. 11F shows another example. In FIG. 11F, two sliders are shown. The first, a linear slider controlled by cursor 190a, allows the patient to adjust the frequency in accordance with frequencies reflected in the optimal volume 200′. A second two-dimensional slider controlled by cursor 190b allows the patient to adjust pulse width and amplitude at that frequency. Preferably, the range of pulse widths and amplitudes is constrained by the optimal parameters 200′ and by the frequency already selected using cursor 190a. For example, if the user selected to use frequency F=400 Hz, the external controller 60 can consult optimal parameters 200′ to automatically determine an optimal range of pulse widths (e.g., 175 to 210 microseconds) and amplitudes (3.7 to 4.1 mA) for the patient to use at that frequency. When the patient changes the frequency using cursor 190a, the range of permissible pulse widths and amplitudes selectable using cursor 190b can automatically change to ensure that sub-threshold stimulation remains within the volume 200′ determined to be statistically useful for the patient.

U.S. Patent Application Publication 2020/0009394, which is incorporated herein by reference in its entirety, discloses other ways in which in which a user can program settings for his IPG 10 using the derived optimal stimulation parameters. As disclosed in that publication, a user interface of the patient's external controller 60 can allow the patent to select from a number of stimulation modes. Such stimulation modes can include various ways in which the IPG can be programmed consistent with optimal stimulation parameters 200 or 200′ determined for the patient, such as: an economy mode that provides stimulation parameters having a low power draw; a sleep mode which optimizes the stimulation parameters for the patient while sleeping; a feel mode which allows a patient to feel the stimulation (supra-perception); a comfort mode for normal everyday use; an exercise mode that provides stimulation parameters appropriate for when the patient is exercising; and an intense mode usable for example if the patient is experiencing pain, and would benefit from more intense stimulation. Such stimulation modes can be indicative of a patient's posture or activity. For example, a sleep mode provides stimulation optimized for sleep (e.g., when the patient is lying down and/or is not moving significantly), and an exercise mode provides stimulation optimized for exercise (e.g., when the patient is standing up and is moving significantly). Stimulation modes can also be included that provide stimulation optimized for different patient postures, such as supine, prone, standing, sitting, etc., or for different conditions such as cold or bad weather. A patient can select from these stimulation modes, and such selections can program the IPG 10 to provide a subset of the stimulation parameters 200 or 200′ useful for that mode, as described in further detail in the '394 Publication. A patient or clinician can also define a custom mode of stimulation. Still further, a stimulation mode can be automatically detected and selected for the patient, again as described in the '394 Publication.

As disclosed in U.S. Patent Application Publication 2020/0147397, which is incorporated herein by reference in its entirety, algorithm 150 can be varied by receiving additional inputs. Such additional inputs can include patient fitting information, such as paresthesia overlap mapping data, perception threshold levels, questionnaire results, etc. Such additional information may be used in the determination of the optimal parameters, or to determine subsets of such parameters.

Algorithm 150 determines a range or volume of optimal stimulation parameters sets 200 or 200', or subsets of such parameters. But not all of these stimulation parameter sets may be equally useful in resolving the patient's symptoms. Therefore, the patient or clinician still must ultimately select one stimulation parameters set (A, PW, F) from this plurality at any given time for use by the patient. This may be difficult for the clinician or patient to navigate using their clinician programmer 70 or external controller 60, given the complexity which with the stimulation parameters sets were determined. FIGS. 10, 11E and 11F provides examples of GUIs to assist with selecting a single stimulation parameter set from within optimal parameters 200 or 200′.

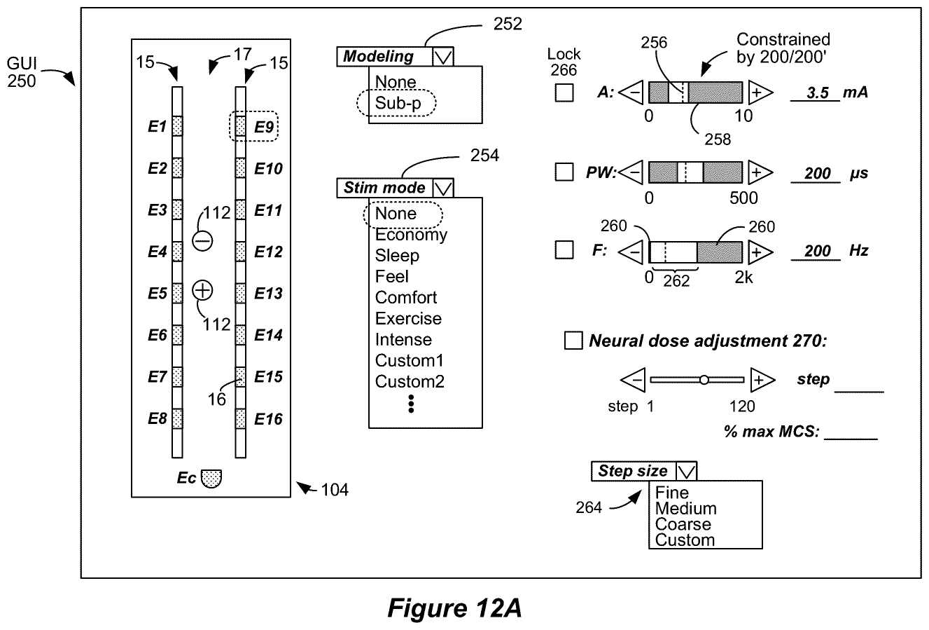

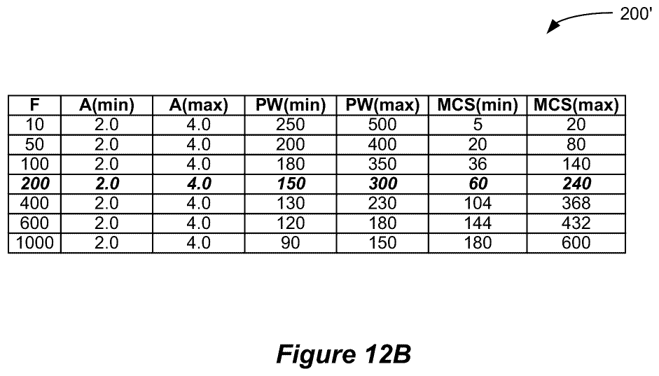

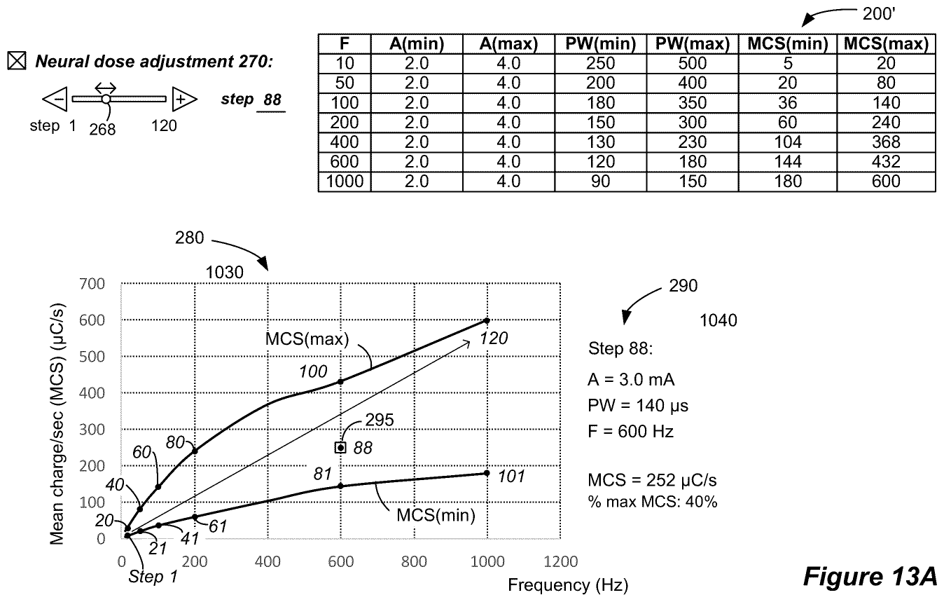

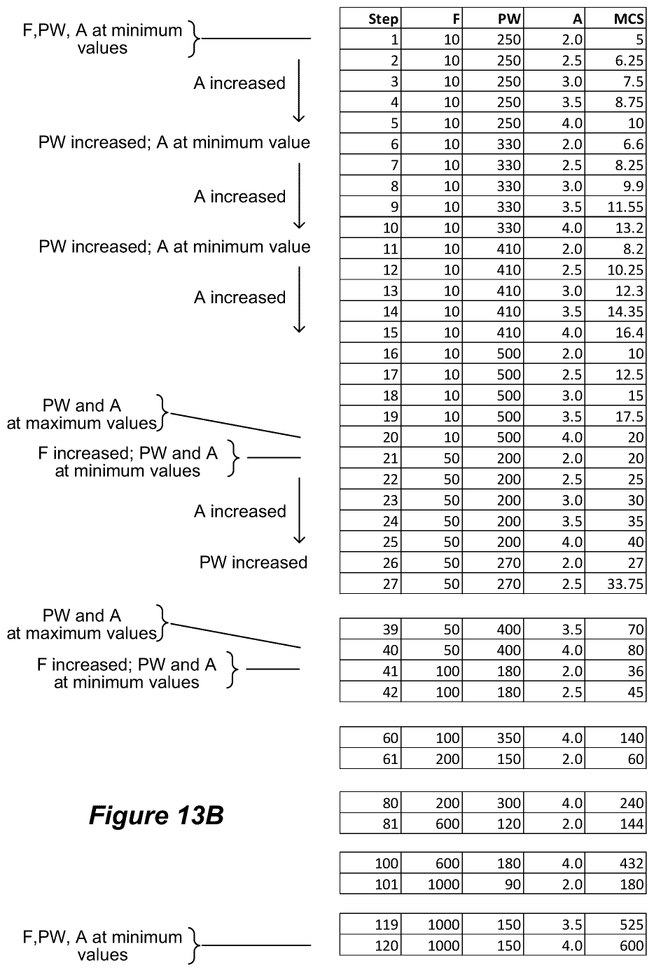

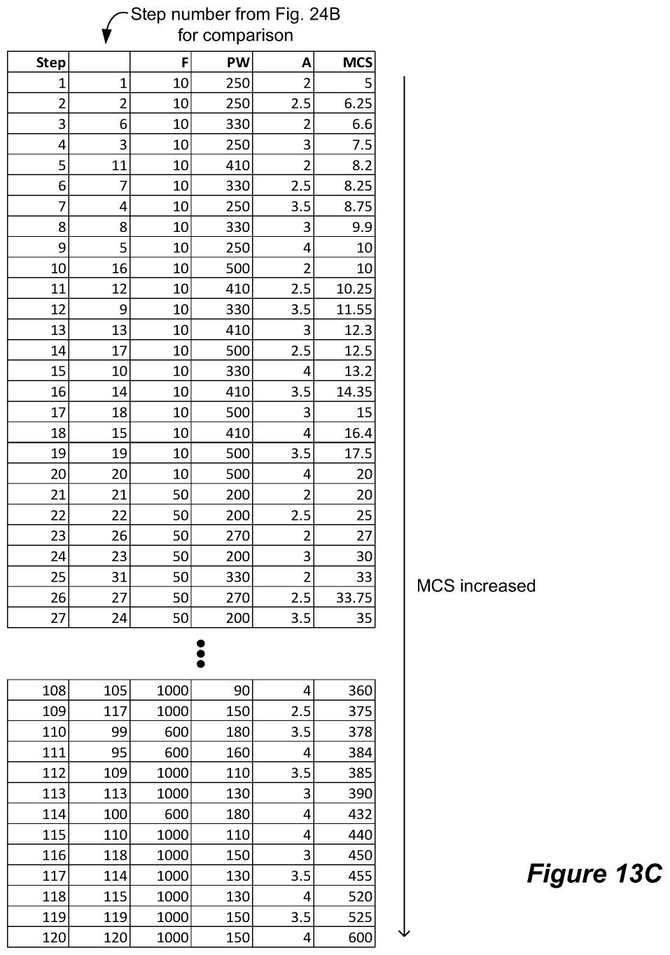

Still, further solutions for navigating optimal parameters 200 or 200′ are disclosed in U.S. Patent Application Publication 2023/0073363, which is incorporated herein by reference. The technique of the '363 Publication is shown here in FIGS. 12A-13C. Starting with FIG. 12A, a graphical user interface GUI 250 is shown, which may be rendered on any external system (see FIG. 4). GUI 250 in this example shows the leads interface 104 including the electrode array 17 and the location of the stimulation (poles 112) in the array. GUI 250 also includes a modeling input 252, which when selected allows the user to select whether sub-perception modeling is to be used in selecting stimulation parameters, or whether no modeling is used. In FIG. 12A, sub-perception (sub-p) stimulation therapy is chosen, which constrains selection of stimulation parameters to parameters sets within the optimal stimulation parameters 200/200' determined earlier (e.g., via algorithm 150). Selection of this option can also cause additional aspects to populate in the GUI. For example, a stimulation mode input 254 may be shown, which may be used to select from the stimulation modes described earlier (economy, sleep, etc.). Finally, a neural dose adjustment interface 270 can also be displayed, and is explained later.

As mentioned, stimulation parameters selectable in FIG. 12A can be constrained by the optimal stimulation parameters 200 or 200′, and in this example simulation parameters 200′ defining a volume of (F,PW,A) coordinates are used. An example of optimal stimulation parameters 200′ is shown in FIG. 12B indexed using frequency, with each frequency being associated with a maximum and minimum pulse width and amplitude. The volume of optimal stimulation parameters 200′ could however be represented differently, as discussed earlier. Note for simplicity in FIG. 12B it is assumed that the amplitude A ranges between a minimum value of 2.0 mA and a maximum value of 4.0 mA for all frequencies, although as noted earlier the modelling and computations may provide different values for the amplitude at different frequencies, and for different patients (especially depending on their perception thresholds pth). The maximum and minimum pulse widths in FIG. 12B by contrast vary between maximum and minimum values consistent with earlier modeling.

Notice in FIG. 12B that the optimal stimulation parameters has been modified to include a maximum and minimum Mean Charge per Second (MCS, in microCoulombs/sec) that the stimulation parameters provide. These MCS values are computable using the maximum and minimum amplitude and pulse width values. Specifically, MCS(min) equals F*A(min)*PW(min), and MCS(max) equals F*A(max)*PW(max). As discussed further below, these MCS values can be a useful means to control the stimulation therapy, and in particular to further constrain selectable stimulation parameters for the patient, as explained further below.

Referring again to FIG. 12A, when sub-p modeling is selected (252), and assuming no stimulation mode is selected (254), the selection of the stimulation parameters is constrained in the GUI 1000 using the optimal stimulation parameters 200′. Further, such constraints can be visualized for the user. In this regard, windows 258 can be provided for each stimulation parameter showing a general range of permissible values (e.g., that the IPG can produce, such as from 0 to 10 mA of amplitude A). Windows 258 can be rendered with exclusion zones 260 representing values for parameters that cannot be selected because they are outside of the range of the optimal stimulation parameters 200′. For example, parameters 200′ specify that only frequencies from 10 to 1000 Hz are appropriate based on the sub-perception modelling and patient measurements taken earlier (as selected at input 252). Thus, the frequency is only adjustable within an inclusion zone 262 between and including these values. Note that an indicator 256 can be displayed within each inclusion zone 262 to indicate the value of each parameter the user has selected (e.g., 200 Hz in the example in FIG. 12A). If particular stimulation modes are selected (254), the GUI 250 may constrain the selectable stimulation parameters even further in accordance with the selected mode, as explained further in the above-referenced '363 Publication.

Other stimulation parameters like amplitude and pulse width can be similarly constrained. In one example, and consistent with the manner in which parameters 200' are indexed (FIG. 12B), the ranges for the parameters in inclusion zones 1012 may dynamically depend on a value selected for one of the stimulation parameters. For example, permissible ranges for the amplitude and pulse width may depend on the frequency that has been selected. For example, and referring to FIG. 12B, at F=200 Hz the pulse width varies between 150 and 300 microseconds, and the amplitude varies between 2.0 and 4.0 mA. Exclusion and inclusion zones 1010 and 1012 are thus rendered in the GUI 250 to limit adjustment of these values given the user's selection of F=200 Hz. In sum, constraining the parameters as shown in FIG. 12A ensures that the user can select stimulation parameters for the patient that are consistent with parameters 200′ determined from modeling. Note that the GUI 250 can also include options 266 to lock certain of the parameters so that they cannot be automatically adjusted. This is useful to keep certain stimulation parameters constant while others may be automatically adjusted. For example, the amplitude can be locked. If the user thereafter adjusts the frequency, the pulse width value and its associated inclusion zone 1012 may be changed, but the amplitude value will not (assuming it is still within its inclusion zone 1012).

GUI 250 as described is helpful in ultimately selecting a particular stimulation parameter set for use by the patient, because constraining selection of the stimulation parameters narrows options to those that should be therapeutically effective for the patient (e.g., to provide sub-perception stimulation). Still, even with options narrowed, the GUI 250 as described so far requires independent selection of each stimulation parameters. This may be difficult, in particular for the patient, to navigate. If therapy is not working well for the patient, should the patient increase the amplitude or the pulse width? Or decrease these? Or change the frequency? These may be complicated questions for the uninformed patient, leading the patient to select different values at random.

This issue is addressed in GUI 250 by the neural dose adjustment interface 270, whose operation is explained with reference to FIGS. 13A-13C. When selected, this interface 270 allows the user to navigate the optimal stimulation parameters 200′ or subsets/modes thereof without selecting those parameters individually. Instead, interface 270 preferably includes selectable step up and step down buttons as shown, which increment though stimulation parameter sets in the optimal stimulation parameters 200′ in a number of steps. In the example shown in FIGS. 13A-13C for example, it is assumed that there are 120 steps, and thus 120 stimulation parameter sets within optimal parameters 200′ that the user can cycle through using the interface 270. The number of steps supported by interface 270 does not necessarily need to correspond to each and every possible stimulation parameter set within optimal parameters 200′ that the IPG is able to provide. In this regard, interface 270 can include an option 264 to adjust the step size (FIG. 12A), which will affect the number of steps, as explained further in the above-referenced '363 Publication. Interface 270 can also include a slider 268 to allow the user to slide through the different steps without using the arrows. Note that the interface 270 can include a check box to activate or deactivate its use. In one example, activating the interface 270 precludes the ability to individually adjust each of the stimulation parameters (e.g., FIG. 12A). Deactivating the interface 270 by contrast allows the stimulation parameters to be individually adjusted in the GUI 250.

FIG. 13A shows operation of the neural dose adjustment interface 270 in a first example. Selection of the interface 270 can include within GUI 250 a graphic 280 that provides the user some idea of how they are navigating the optimal stimulation parameters 200'. In the example shown, graphic 1030 comprises a graph of MCS(min) and MCS(max) versus frequency, which parameters were discussed earlier with reference to FIG. 12B, and which are used to further constrain the selectable stimulation parameters. Displaying information is this manner is useful because it gives the user a sense of how total charge has been constrained, and whether the charge as currently set by the interface 270 is relatively high or low at the current frequency. The GUI 250 may also show information 290 as the interface 270 is stepped through, including the current step number (e.g., 88); the stimulation parameters (A,PW, F) associated with that step; the resulting MCS value of the stimulation (A*PW*F); and how that charge value compares to MCS(max), which is expressed in FIG. 13A as a percentage. The current step can also been shown with a cursor 295 within graphic 280.

Generally speaking, the goal of interface 270 is to step stimulation through different simulation parameters set within the optimal stimulation parameters 200′ as constrained by MCS(max) and MCS(min) at each frequency. The interface 1020 can step through these parameter sets in different ways, but in one example the interface steps the stimulation parameters sets from a lowest MCS value (Step 1) to a highest MCS value (Step 120), as generally indicated by the arrow in graphic 280 in FIG. 13A. FIG. 13B shows one example of how this can occur. At Step 1 a lowest frequency in the optimal stimulation parameters 200′ (10 Hz) is selected, as well as a lowest amplitude (A(min)=2.0 mA) and pulse width (PW(min)=250 μs) at this frequency. See FIG. 12B. If these values cannot be produced by the IPG 10, they may be rounded to values that can while still within the charge limits set by the MCS limits. Such rounding may also occur at subsequent step values.

When the step is incremented (Step 2), the amplitude is increased to its next quantized value (e.g., to 2.5 mA in this example). This continues (Steps 3-4) until the amplitude reaches its maximum (Step 5, A(max)=4.0 mA). At this point, the pulse width can be incremented to its next quantized value (330 μs), with the amplitude set to its lowest value (2.0 mA) (Step 6). The amplitude can then be increased (Steps 7-9) until the maximum value is again reached (Step 10), at which point the pulse width can again be increased, and the amplitude again set to a lowest value (Step 11). This pattern continues until the amplitude and pulse width are maximized (A(max)=4.0 mA and PW(max)=500 μs) at the frequency in question (Step 20).

At this point (Step 21), the frequency is then incremented (e.g., to 50 Hz), and amplitude and pulse widths are set to minimum values at this new frequency (A(min)=2.0 mA and PW(min)=200 μs). The above-described pattern is then repeated at this frequency by increasing the amplitude to its maximum, then increasing the pulse width and minimizing the amplitude, etc., until again a maximum amplitude and pulse width is reached at this frequency (Step 40, A(max)=4.0 mA) and PW(max)=400). The frequency is then incremented (Step 36, F=100 Hz), etc. As this pattern continues, eventually at Step 120 the maximum frequency (1000 Hz), and the maximum amplitude and pulse width at this frequency are reached.

To summarize, the interface 270 steps though stimulation parameter sets with the hope of finding a set that best treats the patient. Assume for example, the patient finds Step 42 to provide the best relief (F=100 Hz, A=2.5 mA, PW=180 microseconds). At this point the user can deselect use of the interface 270 and if desired “tweak” these stimulation parameters individually in the GUI 250, as discussed above with respect to FIG. 12A. At this point the user could increase a decrease the stimulation parameters more finely than allowed by the step size adjustment 264 (e.g., amplitude in 0.1 mA increments, or pulse width in 10 microsecond increments). In any event, a single stimulation parameter set within optimal stimulation parameters 200′ is arrived at more easily and intuitively for the patient.

The graphic 280 of FIG. 13A shows certain of the steps of FIG. 13B. Notice that as the interface 270 and steps are incremented that the mean charge-per-second remains constrained between MCS(max) and MCS(min). Notice further that increasing steps do not necessarily monotonically increase the MCS. For example, Step 61 has a lower MCS value than Step 60. This is because, as shown in FIG. 13B, higher-frequency Step 61 has a lower amplitude and pulse width than lower-frequency Step 60. Of course, the manner in which interface 270 navigates optimal stimulation parameters 200′ is just one example, and the stimulation parameters sets could be navigated in different orders.

FIG. 13C shows another manner in which the interface 270 can navigate the optimal stimulation parameters 200′. In this example, the MCS of the stimulation does monotonically increase with step value, starting at 5 μC/s at Step 1, and steadily increasing to 600 μC/s at Step 120. Essentially, one can understand FIG. 13C as the data of FIG. 13B sorted by increasing MCS values. To understand the differences between these approaches, the step numbers from FIG. 13B are included in FIG. 13C (e.g., what was Step 11 in FIG. 13B is now Step 5 in FIG. 13C). Organizing operation of the interface 270 per FIG. 13C is beneficial because the neural dose of stimulation the patient receives is reliably increased or decreased as the interface is incremented or decremented, even if this results in seemingly random adjustment of the stimulation parameters. For example, in FIG. 13C notice that Step 11 specifies F=10 Hz, PW=410 μs, and A=2.5 mA, while next Step 12 specifies F=10 Hz, PW=330 μs, and A=3.5 mA.

While FIG. 13C shows the various steps/stimulation parameters ordered in accordance with their MCS values, they could be ordered and selected differently. For example, the steps could also be ordered by % max MCS, as described earlier. Further, GUI 250 and neural dose adjustment interface 270 can be used to navigate only a subset of the optimal stimulation parameters 200′ determined previously for the patient. Lastly, while it has been assumed here that the optimal stimulation parameters 200′ used by the GUI 250 and neural dose adjustment interface 270 were determined using the modelling described earlier (FIGS. 6-11F), this is simply one example, and the GUI 250 and neural dose adjustment interface 270 can process optimal stimulation parameters determined by different means.

Optimal sub-perception stimulation parameters, once determined for the patient, may not necessarily need to be constantly applied. It may be beneficial instead to apply such stimulation parameters intermittently to the patient by repeatedly turning on the stimulation for a duration and off for a duration. In other words, the stimulation to the patient can be repeatedly enabled and disabled using a duty cycle. A number of reasons make duty cycling of stimulation a viable option for patients, especially when they are receiving sub-perception stimulation therapy. First, as discussed briefly above, sub-perception stimulation can “wash in” and become therapeutically beneficial to relieve a patient's symptoms quickly, particularly if the location of the stimulation in the electrode array 17 well targets the patient pain, using for example the sweet spot searching technique disclosed in U.S. Pat. No. 10,576,282 referenced above. Second, sub-perception stimulation has also been noted to “wash out” over a significant duration. That is, therapeutic benefit from sub-perception stimulation previously applied to the patient still provides the patient symptomatic relief even after the stimulation has been turned off. Third, duty cycling of the stimulation is beneficial from a power standpoint, because the IPG 10 does not need to expend significant energy when the stimulation is off, which eases demands on the battery 14. Thus, duty cycling of stimulation can extend the life of the IPG 10 (if it has a permanent battery), or make charging of the IPG shorter or less frequent (if it has a rechargeable battery). Fourth, the inventors have noticed that duty cycling of stimulation can prevent over-stimulation and side effects. While the mechanisms of action here are not fully understood, the inventors have noticed empirically that some patients report better results when their sub-perception stimulation is not “free running” and is instead applied with a duty cycle.

While duty cycling of stimulation may be potentially beneficial, establishing that duty cycle may be difficult to establish for a given patient. That is, it may not be obvious how long the stimulation should be on or off for a particular patient, or in light of the stimulation parameters they are using. In this regard, traditional GUIs useable in external systems to program duty cycling of stimulation in an IPG typically do not provide any particular guidance as to how duty cycle on and off times should be set. This makes it difficult for a clinician or patient to select a duty cycling regime, particularly when the GUI allows a wide range of on and off times to be specified.

The inventors have noticed empirically that certain on/off times tend to work well when duty cycling the stimulation of patients receiving sub-perception therapy, and further that these on/off times can vary depending on the particular stimulation parameters being used. For example, on times in a range of 30-60 seconds, and off times in a range from 60-240 seconds, generally perform well for most patients. When lower frequencies are used for the stimulation, which involve the application of smaller neural doses of charge to the patient, higher duty cycles tend to be more therapeutically effective—that is, duty cycling involving longer on times and/or shorter off times. By contrast, when higher frequencies are used involving higher neural doses of charge, lower duty cycles tend to be more therapeutically effective—that is, duty cycling involving shorter on times and/or longer off times. Still further, effective duty cycling can depend on how well a patient responds to sub-perception therapy. If a patient is a good responder to therapy (e.g., reports a significant reduction in symptoms), it may for example be reasonable to use a smaller duty cycle for such patients, meaning their stimulation can be off for a longer percentage of the time. Poorer responders may require their stimulation to be in use more continuously, and hence may require higher duty cycles with shorter off times or longer on times.

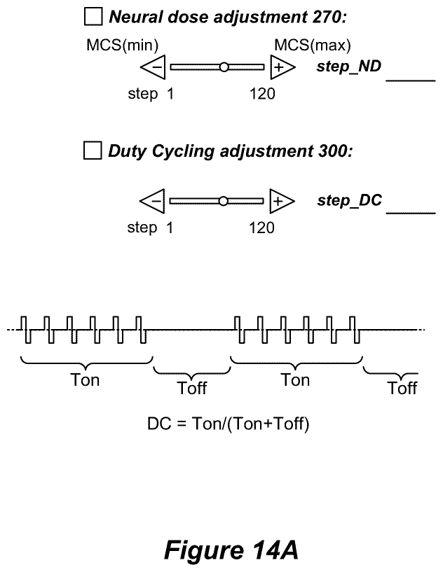

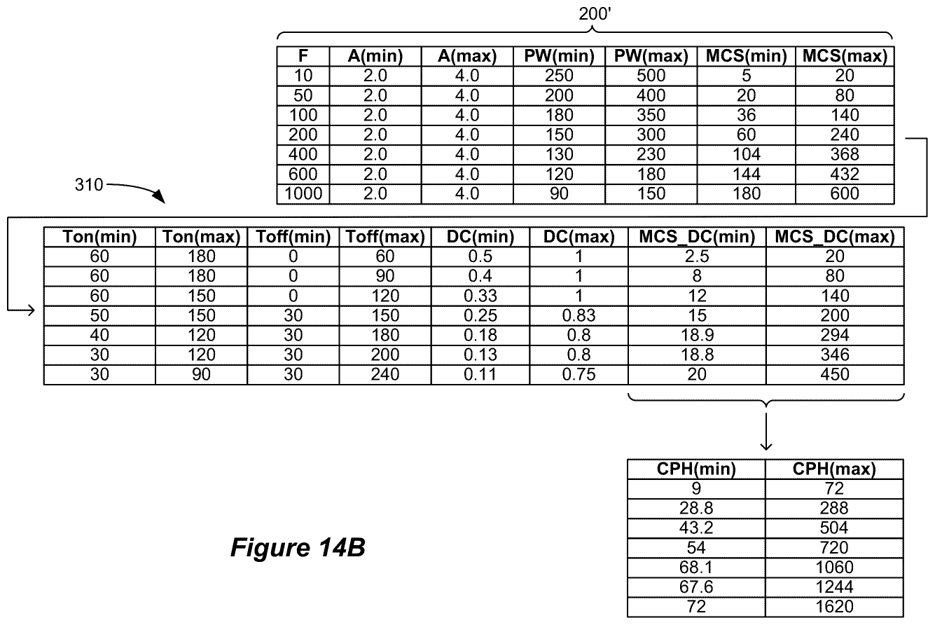

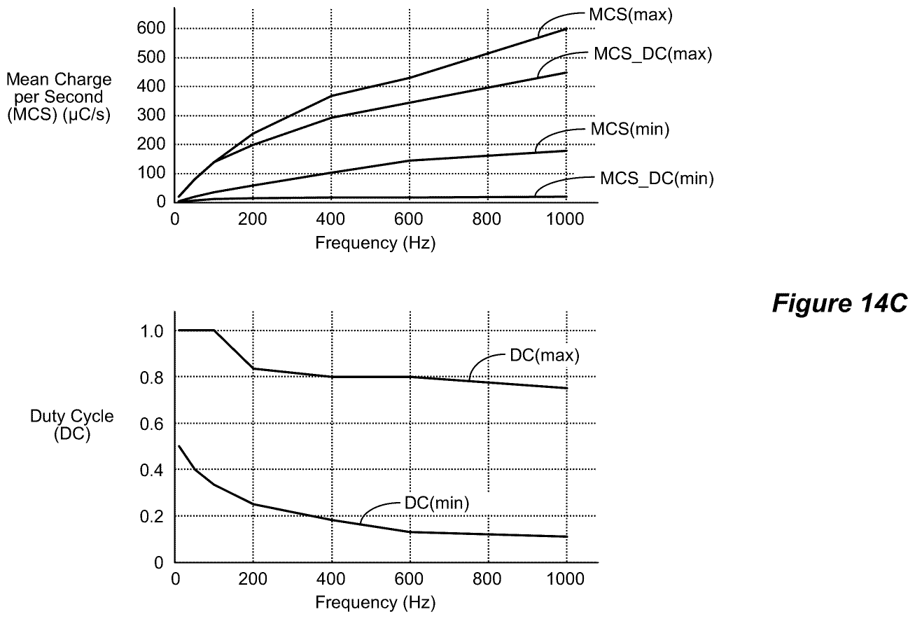

Consistent with these observations, the inventors have devised an improved method for setting the duty cycling of stimulation for patients, which is enabled by an improved GUI and programming of relevant external systems. This improved GUI can be a stand-alone GUI, or can add functionality to the GUI 250 described earlier (FIG. 12A). Significantly, the improved GUI adds a duty cycling adjustment interface 300, as shown in FIG. 14A. Like the neural dose adjustment interface 270 described earlier, the duty cycling adjustment interface 300 preferably includes selectable step up and step down buttons as shown, and/or a slider, to increment or decrement through a series of steps that operate to set the duty cycle of the stimulation otherwise prescribed (e.g., by interface 270). Note in FIG. 14A that these steps are labeled step_DC to differentiate them from neural dose steps step_ND of interface 270 described earlier. Note that the interface 300 can include a check box to activate or deactivate its use. In one example, activating the interface 300 precludes the ability to set the duty cycle (e.g., on and off times) using other means that may be present in GUI 250 (not shown). Deactivating the interface 300 by contrast allows duty cycling to be manually set elsewhere in the GUI 250.

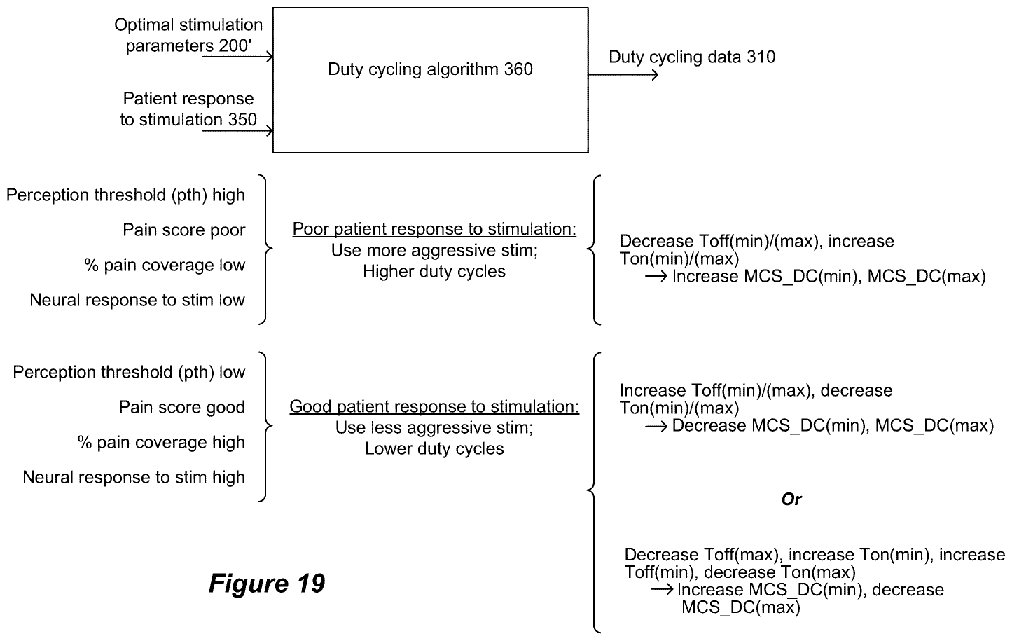

As discussed, the duty cycling adjustment interface 300 applies a duty cycling to the otherwise prescribed stimulation parameters (270), which is affected by cycling stimulation with an on time (Ton) and an off time (Toff), as shown in the waveform in FIG. 14A. Example values for Ton and Toff are discussed further below, but are typically in a much narrower and more therapeutically-useful range compared to what the GUI of the external device can otherwise permit. For example, whereas Ton and Toff can generally be set in the GUI from about 10 seconds to 90 minutes, these values are set by interface 300 to times on the order of tens of seconds to minutes in the examples described below. Preferably, the clinician or patient need not individually select durations for Ton and Toff, as these are instead automatically set by the interface 300 at each step and are determined by the external system in a manner consistent with observations noted earlier. In this regard, use of the interface 300 can be supported by duty cycling data 310 as shown in FIG. 14B. Preferably, the duty cycling data 310 is based in part on the optimal stimulation parameters 200′ determined earlier for the patient. Like parameters 200′, duty cycling data 310 can be determined and stored in a relevant external system. For example, a relevant external system can include a duty cycling algorithm 360 (e.g., part of software 96, FIG. 4) to determine the duty cycling data 310 from the optimal stimulation parameters 200′, as shown in FIG. 19. Like the optimal stimulation parameters 200′, the duty cycling data 310 once determined can be transmitted to a patient external controller 60 to set the operation of the interface 300 for the patient.

In the example shown, the optimal parameters 200′ comprise a volume of potential (F, A, PW) coordinates, which is reflected as maximum/minimum amplitudes (A(max), A(min)) and maximum/minimum pulse widths (PW(max), PW(min)) for frequencies (F) of interest. These optimal parameters are preferably determined as described earlier, although the optimal stimulation parameters 200′ can be determined in different manners and using different techniques from those described earlier. From these values, a maximum/minimum Mean Charge per Second (MCS(max), MCS(min)) can be computed at each frequency as explained earlier. Duty cycling data 310, via operation of algorithm 360, preferably adds to such data suitable on and off time ranges at each frequency, which are specified using minimum and maximum values (Ton(min), Ton(max), Toff(min), Toff(max)). These values are set by the algorithm 360 consistent with duty cycling observations discussed earlier.

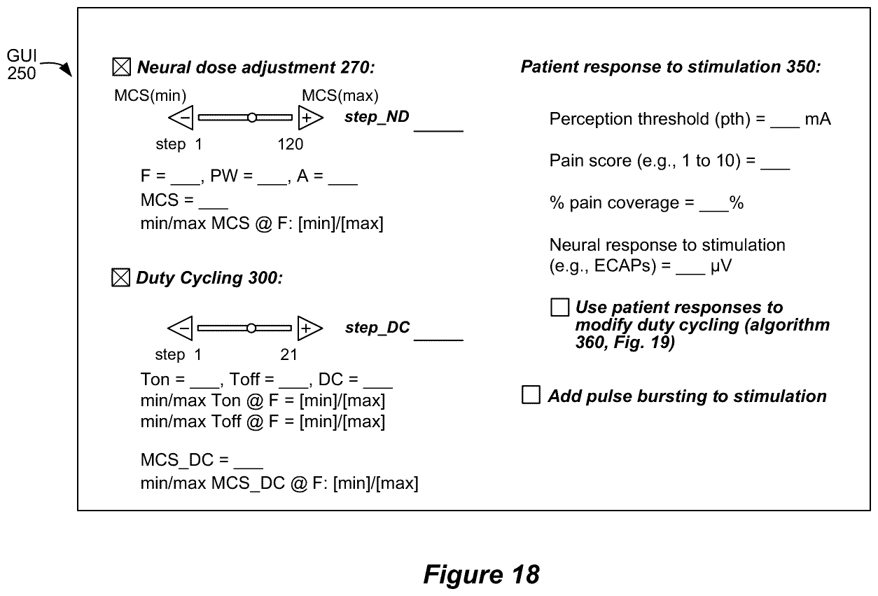

For example, Ton is set minimally to values of 30 to 60 seconds, and is set maximally to values of 90 to 180 seconds, noticed to be generally effective. Toff is set minimally to values from 0 to 30 seconds, and is set maximally to values of 60 to 240 seconds, also noticed to be generally effective. These ranges/values for Ton and Toff may be varied depending on patient's responses to stimulation (see FIGS. 18 and 19), but this is explained later. Furthermore, using these settings, lower frequencies generally have higher duty cycles than higher frequencies. This can be seen by looking at the minimum and maximum duty cycles (DC(min), DC(max)) at each frequency, where DC(min) is computed using Ton(min) and Toff (max) (DC(min)=Ton(min)/(Ton(min)+Toff(max)), and DC(max) is computed using Ton(max) and Toff (min) (DC(max)=Ton(max)/(Ton(max)+Toff(min)). Thus, at a low frequency such as 10 Hz, the duty cycle ranges from 0.5 to 1 (with ‘1’ meaning no duty cycling is used), whereas at a high frequency such as 1000 Hz, the duty cycle is lower ranging from 0.11 to 0.75. In other words, the plurality of selectable duty cycles (as defined by DC(max) and DC(min) have values that decrease as the selected frequency increases. DC(min) and DC(max) are shown graphically as a function of frequency in FIG. 14C.

Duty cycling data 310 also includes maximum and minimum Mean Charge per Second values when duty cycling is used (MCS_DC(min), MCS_DC(max)), where MCS_DC(min) equals the minimum MCS of the stimulation with minimum duty cycling (MCS(min))*DC(min), and where MCS_DC(max)=MCS(max)*DC(max). MCS_DC(max) and MCS_DC(max) are also graphed as a function of frequency in FIG. 14C, along with MCS(max) and MCS(min) for the optimal stimulation parameters 200′ when duty cycling isn't used. As in earlier examples, this average charge rate is expressed in FIG. 14C in microCoulombs/second, although both the charge and time basis of this charge rate can be changed. For example, and as shown in FIG. 14B, the average charge rate can be expressed as a mean Charge-Per-Hour (CPH) expressible in milliCoulombs/hour, which may comprise a more meaningful metric to express an average charge rate in which stimulation is turned on and off on the order of minutes. Therefore, MCS_DC(min) and MCS_DC(max) can respectively be expressed as CPH(min) and CPH(max) once appropriate conversions are made to charge (1000 microCoulombs equals milliCoulombs) and to time (3600 seconds equals one hour). The average charge rate when duty cycling is used (and its maximum and minimum values) can be expressed using other meaningful units as well. That being said, subsequent discussion will discuss an average charge rate in terms of MCS (expressed in microCoulombs/second).

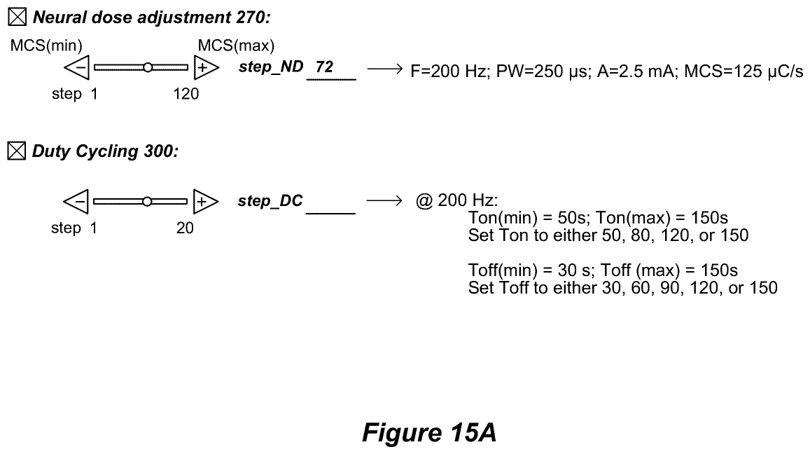

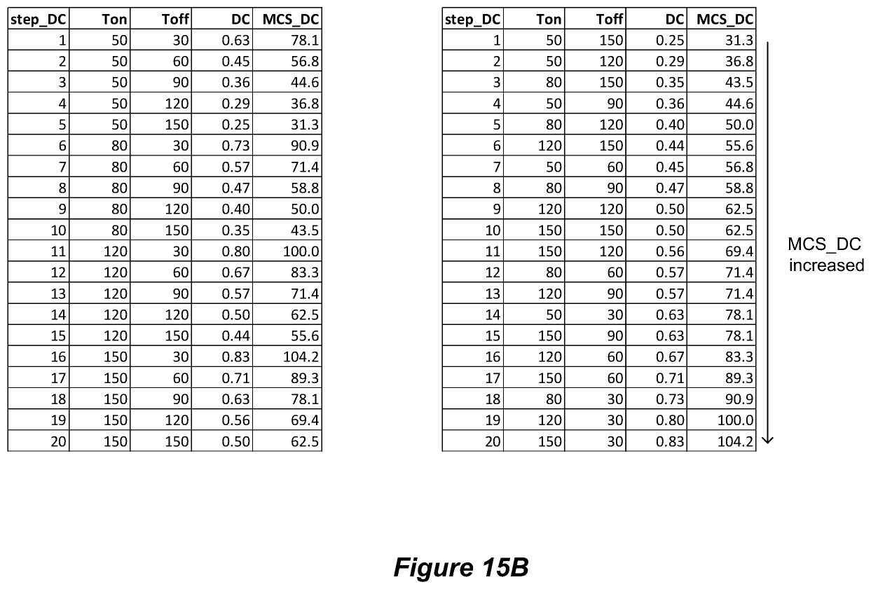

With duty cycling data 310 so established in the relevant system, operation of duty cycling adjustment interface 300 can be better understood, and is explained in a first example in FIGS. 15A and 15B. In this example, a clinician or patient selects use of a particular optimal stimulation parameter 200′ using the neural does adjustment interface 270, as explained earlier, and in this example it is assumed that parameters are select at step_ND=72—i.e., F=200 Hz, PW=250 μs, and A=2.5 mA, which yields MCS=125 μC/s. Choosing the stimulation parameters affects the duty cycling options that duty cycling adjustment interface 300 can provide. While dependence of the duty cycling on the stimulation parameters could be implemented in different ways, it is assumed here that the frequency selected for stimulation (F=200 Hz) will dictate the duty cycling by limiting the on and off times to those specified in duty cycling data 310 at this frequency—i.e., Ton(min)=50 s, Ton(max)=150 s, Toff(min)=30 s, and Toff(max)=150 s. As noted earlier, this is sensible, because frequency in particular has been noticed as a parameter upon which duty cycling should depend. When duty cycling is used in conjunction with the selected parameters, an MCS value results (MCS_DC) which will necessarily be lower than the MCS value for the parameters (125 μC/s) when duty cycling is not used, as explained further below.

Once these minimum/maximum values for Ton and Toff have been determined, the external system can populate potential values for Ton and Toff within these ranges. Such potential values can depend on the number of steps (step_DC), and may further depend on the values for Ton and Toff that the GUI 250 is otherwise capable for producing. In the simple example shown in FIG. 15A, it is assumed that the external device has selected values for Ton of either 50s (Ton(min)), 80s, 120s, or 150 s (Ton(max)), and values for Toff of 30s (Toff(min), 60s, 90s, 120s, or 150 s (Toff(max)). As such, there are 20 different combinations of Ton and Toff values, and thus 20 steps (step_DC) that the GUI 250 will populate for selection in interface 300.

FIG. 15B shows potential manners in which a patient may change the duty cycling of their stimulation using duty cycling adjustment interface 300, and the effect of such duty cycle changes on the charge the patient receives (MCS_DC). To the left, various steps (step_DC from 1 to 20) move through the different combinations of Ton and Toff, e.g., by setting Ton to 50, then incrementing Toff (step_DC 1-5), then incrementing Ton to 80 and repeating the Toff values (step_DC 6-10), etc. Notice that this doesn't result in monotonically increasing the MCS_DC that is delivered to the patient. By contrast, the right shows the resulting combinations of Ton and Toff ordered by MCS_DC, with step_DC=1 delivering a minimum charge, and step_DC=20 delivering a maximum charge. This may comprise a more-intuitive ordering of the steps in interface 300, as it may be viewed by the patient as a sort of charge, intensity, or energy slider.

Although not shown, it should be understood that operation of duty cycling adjustment interface 300 can change as the stimulation parameters are changed, i.e., as the neural dose adjustment interface 270 is changed. New stimulation parameters (in particular new frequencies), may cause Ton and Toff min/max to change, which will in turn can change the duty cycling that interface 300 provides on a step-by-step basis.

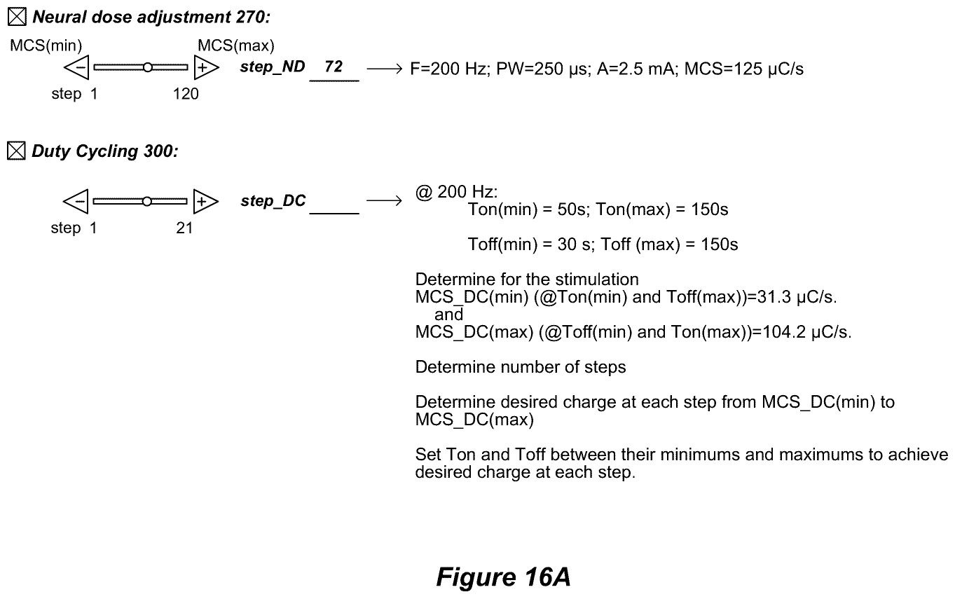

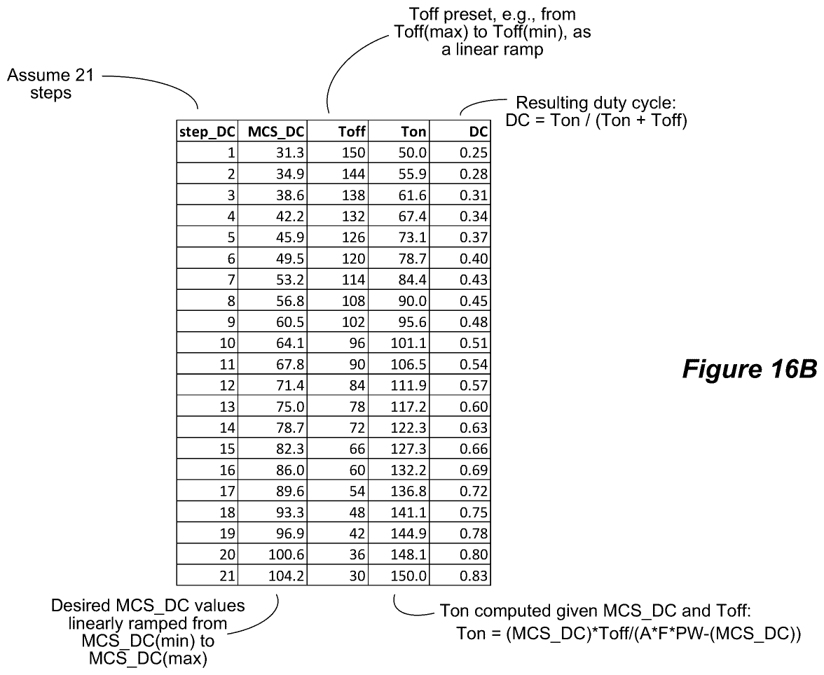

FIGS. 16A and 16B show another example of how duty cycling adjustment interface 300 data may implemented. In this example, desired MCS_DC values and steps to be used during duty cycling are determined given the stimulation parameters, with the external system then determining suitable durations for Ton and Toff to affect appropriate duty cycles at each step. This is described in a flow chart form in FIG. 16A, and various resulting steps (steps_DC) are shown in FIG. 16B. In this example, max and min values for Ton and Toff are determined for the stimulation parameters in question using duty cycling data 310 (FIG. 14A). Thus, at F=200 Hz, Ton(min)=50 s, Ton(max)=150 s, Toff(min)=30 s, and Toff(max)=150 s. Next, MCS_DC(min) and MCS_DC(max) are determined for the stimulation selected using the neural dose adjustment interface 270: at F=200 Hz, PW=250 μs, and A=2.5 mA, MCS_DC(min)=A*F * PW*Ton(min)/(Ton(min)+Toff(max)), which equals 31.3 μC/s, and MCS_DC(max)=A*F * PW*Ton(max)/(Ton(max)+Toff(min)), which equals 104.2 μC/s.

Next a number of steps (step_DC) are determined, which in this example is set to 21. Although not shown, the number of steps could also be entered in the GUI 250. Once the number of steps is determined, desired MCS_DC values at each step can be computed, and as shown in FIG. 16B, these MCS_DC values may comprise a linear ramp from MCS_DC(min) to MCS_DC(max). Alternatively, the number of steps to could be automatically determined based on a desired MCS_DC resolution. For example, the user may input a MCS_DC resolution of 2 μC/s for example (yielding desired MCS_DC values of 31.3, 33.3, 35.3, etc.), with the external system then computing the number of steps needed between MCS_DC(min) and MCS_DC(max) (e.g., (104.2-31.3)/2 equals approximately 36 steps).

From these desired MCS_DC values, the duty cycling at each step—i.e., Ton and Toff—is determined that will provide the desired MCS_DC value. Determining the duty cycle at each step can occur in numerous different ways, but in the depicted example, this occurs by presetting values for Toff at each of the steps, starting at Toff(max), and linearly ramping to Toff(min) at the last step. Ton at each step can then be computed for each step in accordance with the equation shown in FIG. 16B. Notice that Ton calculated in this manner will range from Ton(min) to Ton(max), although it may not be exactly linear. If necessary, values for Ton and Toff can be rounded to values the external system and/or the IPG 10 are capable of producing. Although not shown, one skilled will understand that values for Ton could also be preset, and values for Toff computed.

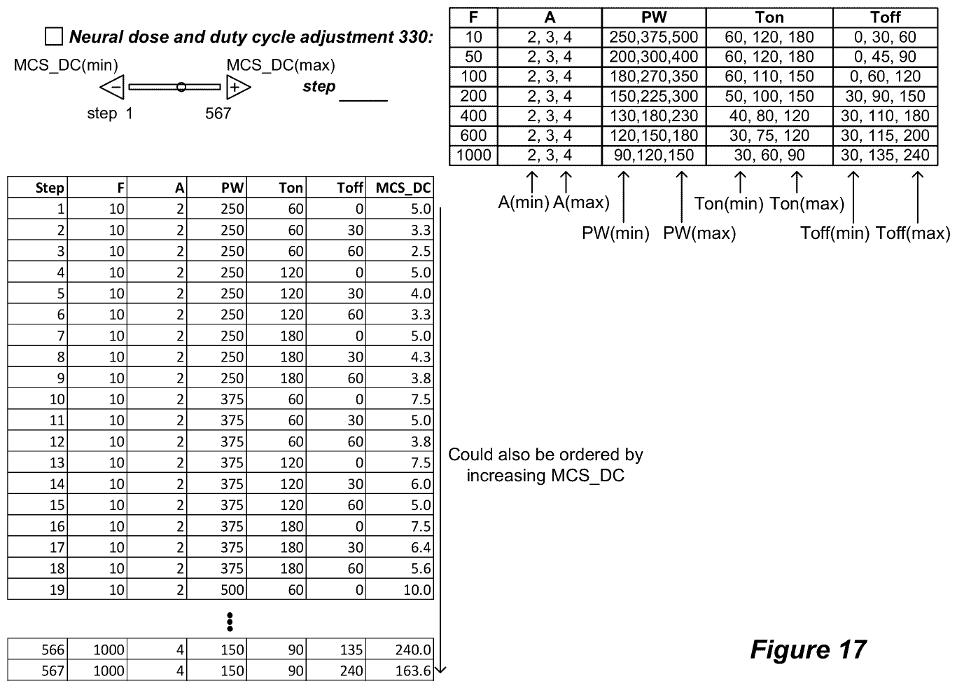

FIG. 17 shows another example of how duty cycling may be implemented in conjunction with optimal stimulation parameters 200′. In this example, a single neural dose and duty cycling adjustment interface 330 is used to simultaneously set or adjust both a patient's stimulation parameters and a duty cycle in accordance with the optimal stimulation parameters 200′ and duty cycling data 310. Specifically, different combinations of the optimal stimulation parameters 200′ are combined with different combinations of Ton and Toff values from the duty cycling data 310. This is shown at the top of FIG. 17 in tabular form. Included in this table are stimulation certain parameters from optimal stimulation parameters 200′ (A, PW) as well as Ton and Toff data from duty cycling data 310, including minimum and maximum values for these parameters at each frequency. Certain other values for these parameters between the minimum and maximum may also be populated, such as midpoints between the minima and maxima. Thus, in total, this example shows three values for each parameter at each frequency, although there could be more.

Different combinations of these parameters are determined and populated as steps in the interface 330, and in this example, three different values for the four parameters A, PW, Ton, and Toff and the seven different frequencies yields a total of 4*4*4*4*7=567 steps. Initial of these steps are shown at the left in FIG. 17, starting with A(min), PW(min), Ton(min) and Toff(min) at the lowest frequency (step 1), and ending with A(max), PW(max), Ton(max) and Toff(max) at the highest frequency (step 567). The steps can be ordered differently, and as in other examples can be sorted by increasing MCS values, which causes the MCS_DC values to range between MCS_DC(min) and MCS_DC(max), as described earlier with respect to FIG. 14A. This example is particularly useful because patient adjustment to stimulation is made easier, as there is only one interface 330 to adjust which will both select optimal stimulation parameters and optimal duty cycling at those stimulation parameters for the patient.

FIG. 18 shows a further example of GUI 250 and how it may present the interfaces 270 and 300 along with other useful information. Neural dose adjustment interface 270 can display information such as the step number (step_ND), the optimal stimulation parameters at that step (F, PW, A) and the MCS (F*PW*A) for those parameters. The interface 270 may display MCS(min) and MCS(max) at the selected frequency to provide an indication of how significant the current MCS value is. The duty cycling adjustment interface 300 may display similarly useful information such as the step number (step_DC), the current values of Ton and Toff, which in turn determines the duty cycle (DC). The interface 300 may display Ton and Toff minimum and maximum values at the selected frequency (from 310) to again provide an indication of how significant the current duty cycling is. Although not shown, note that these and other parameters could also be displayed graphically, for example, as a function of frequency. For example, currently selected settings can be graphically bounded between relevant minimum and maximum values.

As noted earlier, effective duty cycling can depend on how well a patient responds to sub-perception therapy, and in a further modification to FIG. 18, duty cycling can be adjusted in response to input regarding such patient responses. This is shown in interface 350, which allows the clinician or patient to enter information indicative of how well the patient is responding to sub-perception stimulation therapy.

Interface 350 can receive input indicative of a number of pieces of information. For example, the patient's perception threshold (pth) can indicate how well stimulation is targeting the patient's neural tissue, with lower scores indicating better targeting. A pain score derived based on subjective feedback from the patient can also be used, as well as the patient's subjective estimate for how well stimulation is covering his pain (shown in FIG. 18 as a percentage). Pain scores and percent coverage can also be combined into a single metric, as described further in U.S. Patent Application Publication 2022/0387808, which is incorporated by reference in its entirety. Objective measures of clinical efficacy can also be provided at interface 350. For example, a neural response to stimulation, such as an Evoked Compound Action Potential (ECAPs), can be sensed and measured. Sensing of ECAPs is discussed further in U.S. Patent Application Publication 2020/0305744, which is incorporated by reference, and any relevant ECAP feature (such as a peak-to-peak height) can be entered at interface 350. If various parameters are input at 350, the external system may average them to determine a single metric indicative of the patient's response to stimulation.

The one or more patient responses at interface 350 can be used in various ways by the duty cycling algorithm 360 of FIG. 19 to modify the patient's stimulation, and in particular to modify the duty cycling. Such modification can come in many forms, but FIG. 19 generally considers modifications that can be made when input(s) to interface 350 indicate a poor or good responder to stimulation therapy. A poor responder may require more aggressive stimulation therapies and may require higher duty cycling such that the resulting stimulation is on for relatively longer periods of time. This increases the overall charge the patient receives. A good responder may tolerate less aggressive stimulation therapies and may tolerate lower duty cycling such that the resulting stimulation is on for relatively shorter periods of time. This reduces the overall charge the patient receives, which may also beneficially protect the patient from overstimulation and reduce power requirements in the IPG.

If a patient is a poor responder to therapy as evidenced by input(s) received at interface 350, it may be reasonable to use higher duty cycles for such patients—i.e., shorter off times, and/or longer on times—such that stimulation is active for a greater percentage of time. Thus, as shown in FIG. 19, the algorithm 360 may decrease Toff(min) or Toff(max) or both; and/or may increase Ton(min) or Ton(max), or both. These actions would increase the duty cycles selectable via interfaces 300 or 330, and hence increase the charge delivered to the patient (increasing MCS_DC(min) and/or MCS_DC(max)). The algorithm 360 could also adjust the stimulation parameters (e.g., at interface 270) to another value within optimal stimulation parameters 200′ that provides more aggressive stimulation, such as by increasing A, F, or PW.

By contrast, if a patient is a good responder to therapy as evidenced by input(s) received at interface 350, it may be reasonable to use lower duty cycles for such patients—i.e., longer off times, and/or shorter on times—such that stimulation is active for a lesser percentage of time. Thus, as shown in FIG. 19, the algorithm 360 may increase Toff(min) or Toff(max) or both; and/or may decrease Ton(min) or Ton(max), or both. These actions would decrease the duty cycles selectable at interfaces 300 or 330, and hence decrease the charge delivered to the patient (decreasing MCS_DC(min) and/or MCS_DC(max)). The algorithm 360 could also adjust the stimulation parameters (e.g., at interface 270) to another value within optimal stimulation parameters 200′ that provides less aggressive stimulation, such as by decreasing A, F, or PW.

Alternatively, the algorithm 360 may restrict duty cycling variability for a good responder. For example, the algorithm 260 may decrease Toff(max) and/or Ton(max), and/or increase Ton(min) and/or Toff(min). This has the effect of decreasing MCS_DC(max), or increasing MCS_DC(min), or both, which effectively reduces the range of duty cycle values (DC) that are selectable at interfaces 300 or 330. This may be reasonable for a good responder, because a good responder may not require as wide a range of duty cycling options to achieve effective sub-perception stimulation therapy.

Various aspects of the disclosed techniques, including processes implementable in the IPG, or in external system such as the clinician programmer or external controller to render and operate the GUI 250, can be formulated and stored as instructions in a computer-readable media associated with such devices, such as in a magnetic, optical, or solid state memory. The computer-readable media with such stored instructions may also comprise a device readable by the clinician programmer or external controller, such as in a memory stick or a removable disk, and may reside elsewhere. For example, the computer-readable media may be associated with a server or any other computer device, thus allowing instructions to be downloaded to the clinician programmer system or external controller or to the IPG, via the Internet for example. Methods involving use of the disclosed subject matters also comprise aspects of Applicant's invention.

While the disclosed techniques have been illustrated in the context of a spinal cord stimulation system, they may also be implemented in other neurostimulator systems as well (e.g., Deep Brain Stimulator (DBS) systems), or implantable stimulation systems more generally. Furthermore, while the disclosed techniques have been described as being particularly useful in the context of providing sub-perception stimulation, the disclosed techniques may also be applied to other stimulation regimes, such as supra-perception stimulation regimes involving paresthesia.

Although particular embodiments of the present invention have been shown and described, it should be understood that the above discussion is not intended to limit the present invention to these embodiments. It will be obvious to those skilled in the art that various changes and modifications may be made without departing from the spirit and scope of the present invention. Thus, the present invention is intended to cover alternatives, modifications, and equivalents that may fall within the spirit and scope of the present invention as defined by the claims.

What is claimed is:

1. A method for controlling an implantable stimulator device of a patient using an external device in communication with the implantable stimulator device, the method comprising:
receiving at a graphical user interface (GUI) of the external device a selection of a set of stimulation parameters for the implantable stimulator device to execute to provide stimulation to the patient; and
providing on the GUI a first interface to select from one of a plurality of duty cycles for the stimulation, wherein each duty cycle comprises an on duration and an off duration at which the stimulation will be repeatedly enabled and disabled, wherein the plurality of selectable duty cycles are dependent on the selected set of stimulation parameters.

2. The method of claim 1, wherein the selected set of stimulation parameters defines the stimulation as a sequence of stimulation pulses.

3. The method of claim 2, wherein the selected set of stimulation parameters comprises a frequency of the stimulation pulses.

4. The method of claim 3, wherein the plurality of selectable duty cycles are dependent on the frequency of the selected set of stimulation parameters.

5. The method of claim 4, wherein the plurality of selectable duty cycles have values that decrease as the frequency of the selected set of stimulation parameters increases.

6. The method of claim 1, wherein the first interface is configured to allow a user to step though the plurality of duty cycles.

7. The method of claim 6, wherein the first interface is configured to step through the plurality of duty cycles from a minimum duty cycle to a maximum duty cycle.

8. The method of claim 7, wherein the minimum duty cycle is defined by a minimum charge rate, and wherein the maximum duty cycle is defined by a maximum charge rate.

9. The method of claim 1, wherein the plurality of duty cycles are defined using duration parameters including a minimum on duration, a maximum on duration, a minimum off duration, and a maximum off duration, wherein the duration parameters are dependent on the selected set of stimulation parameters.

10. The method of claim 9, wherein the plurality of duty cycles are defined using different combinations of on durations bounded between the minimum and maximum on durations, and off durations bounded between the minimum and maximum off durations.

11. The method of claim 1, wherein the selection of the set of stimulation parameters is received at a second interface provided on the GUI.

12. The method of claim 11, wherein the second interface is configured to allow a user to step though a plurality of sets of stimulation parameters to select the set of stimulation parameters.

13. The method of claim 12, wherein each of the plurality of sets of stimulation parameters comprises a frequency, an amplitude, and a pulse width of the stimulation.

14. The method of claim 12, wherein the plurality of sets of stimulation parameters are derived from a model for the patient determined from providing test stimulation to the patient.

15. The method of claim 14, further comprising providing the test stimulation to the patient to determine the model.

16. The method of claim 11, wherein each of the sets of stimulation parameters comprises a charge rate, wherein the second interface is configured to step through the plurality of sets of stimulation parameters from a minimum charge rate to a maximum charge rate.

17. The method of claim 1, further comprising receiving at the GUI at least one indication of a response from the patient to the stimulation.

18. The method of claim 17, wherein the plurality of selectable duty cycles are further dependent on the at least one indication of the response.

19. The method of claim 1, wherein the stimulation comprises sub-perception stimulation.

20. An external device for controlling an implantable stimulator device of a patient, comprising:
a graphical user interface (GUI) configured to allow a user to select a set of stimulation parameters for the implantable stimulator device to execute to provide stimulation to the patient;
wherein the GUI comprises a first interface configured to allow the user to select from one of a plurality of duty cycles for the stimulation, wherein each duty cycle comprises an on duration and an off duration at which the stimulation will be repeatedly enabled and disabled, wherein the plurality of selectable duty cycles are dependent on the selected set of stimulation parameters.
